SEQUENCE LISTING (2 sequences)

SEQ ID NO: 1 - DNA, 5760 bp, Homo sapiens
aaagatcgctgtgcagtcagccttaaacactgactgcacccctcccagatttcttttacattaactaaaaagtcttatcacacaatctcataaaatttatgtaatttcatttaattttagccacaaatcatcaaaatgacgaggattttgacagctttcaaagtggtgaggacactgaagactggttttggctttaccaatgtgactgcacaccaaaaatggaaattttcaagacctggcatcaggctcctttctgtcaaggcacagacagcacacattgtcctggaagatggaactaagatgaaaggttactcctttggccatccatcctctgttgctggtgaagtggtttttaatactggcctgggagggtacccagaagctattactgaccctgcctacaaaggacagattctcacaatggccaaccctattattgggaatggtggagctcctgatactactgctctggatgaactgggacttagcaaatatttggagtctaatggaatcaaggtttcaggtttgctggtgctggattatagtaaagactacaaccactggctggctaccaagagtttagggcaatggctacaggaagaaaaggttcctgcaatttatggagtggacacaagaatgctgactaaaataattcgggataagggtaccatgcttgggaagattgaatttgaaggtcagcctgtggattttgtggatccaaataaacagaatttgattgctgaggtttcaaccaaggatgtcaaagtgtacggcaaaggaaaccccacaaaagtggtagctgtagactgtgggattaaaaacaatgtaatccgcctgctagtaaagcgaggagctgaagtgcacttagttccctggaaccatgatttcaccaagatggagtatgatgggattttgatcgcgggaggaccggggaacccagctcttgcagaaccactaattcagaatgtcagaaagattttggagagtgatcgcaaggagccattgtttggaatcagtacaggaaacttaataacaggattggctgctggtgccaaaacctacaagatgtccatggccaacagagggcagaatcagcctgttttgaatatcacaaacaaacaggctttcattactgctcagaatcatggctatgccttggacaacaccctccctgctggctggaaaccactttttgtgaatgtcaacgatcaaacaaatgaggggattatgcatgagagcaaacccttcttcgctgtgcagttccacccagaggtcaccccggggccaatagacactgagtacctgtttgattcctttttctcactgataaagaaaggaaaagctaccaccattacatcagtcttaccgaagccagcactagttgcatctcgggttgaggtttccaaagtccttattctaggatcaggaggtctgtccattggtcaggctggagaatttgattactcaggatctcaagctgtaaaagccatgaaggaagaaaatgtcaaaactgttctgatgaacccaaacattgcatcagtccagaccaatgaggtgggcttaaagcaagcggatactgtctactttcttcccatcacccctcagtttgtcacagaggtcatcaaggcagaacagccagatgggttaattctgggcatgggtggccagacagctctgaactgtggagtggaactattcaagagaggtgtgctcaaggaatatggtgtgaaagtcctgggaacttcagttgagtccattatggctacggaagacaggcagctgttttcagataaactaaatgagatcaatgaaaagattgctccaagttttgcagtggaatcgattgaggatgcactgaaggcagcagacaccattggctacccagtgatgatccgttccgcctatgcactgggtgggttaggctcaggcatctgtcccaacagagagactttgatggacctcagcacaaaggcctttgctatgaccaaccaaattctggtggagaagtcagtgacaggttggaaagaaatagaatatgaagtggttcgagatgctgatgacaattgtgtcactgtctgtaacatggaaaatgttgatgccatgggtgttcacacaggtgactcagttgttgtggctcctgcccagacactctccaatgccgagtttcagatgttgagacgtacttcaatcaatgttgttcgccacttgggcattgtgggtgaatgcaacattcagtttgcccttcatcctacctcaatggaatactgcatcattgaagtgaatgccagactgtcccgaagctctgctctggcctcaaaagccactggctacccattggcattcattgctgcaaagattgccctaggaatcccacttccagaaattaagaacgtcgtatccgggaagacatcagcctgttttgaacctagcctggattacatggtcaccaagattccccgctgggatcttgaccgttttcatggaacatctagccgaattggtagctctatgaaaagtgtaggagaggtcatggctattggtcgtacctttgaggagagtttccagaaagctttacggatgtgccacccatctatagaaggtttcactccccgtctcccaatgaacaaagaatggccatctaatttagatcttagaaaagagttgtctgaaccaagcagcacgcgtatctatgccattgccaaggccattgatgacaacatgtcccttgatgagattgagaagctcacatacattgacaagtggtttttgtataagatgcgtgatattttaaacatggaaaagacactgaaaggcctcaacagtgagtccatgacagaagaaaccctgaaaagggcaaaggagattgggttctcagataagcagatttcaaaatgccttgggctcactgaggcccagacaagggagctgaggttaaagaaaaacatccacccttgggttaaacagattgatacactggctgcagaatacccatcagtaacaaactatctctatgttacctacaatggtcaggagcatgatgtcaattttgatgaccatggaatgatggtgctaggctgtggtccatatcacattggcagcagtgtggaatttgattggtgtgctgtctctagtatccgcacactgcgtcaacttggcaagaagacggtggtggtgaattgcaatcctgagactgtgagcacagactttgatgagtgtgacaaactgtactttgaagagttgtccttggagagaatcctagacatctaccatcaggaggcatgtggtggctgcatcatatcagttggaggccagattccaaacaacctggcagttcctctatacaagaatggtgtcaagatcatgggcacaagccccctgcagatcgacagggctgaggatcgctccatcttctcagctgtcttggatgagctgaaggtggctcaggcaccttggaaagctgttaatactttgaatgaagcactggaatttgcaaagtctgtggactacccctgcttgttgaggccttcctatgttttgagtgggtctgctatgaatgtggtattctctgaggatgagatgaaaaaattcctagaagaggcgactagagtttctcaggagcacccagtggtgctgacaaaatttgttgaaggggcccgagaagtagaaatggacgctgttggcaaagatggaagggttatctctcatgccatctctgaacatgttgaagatgcaggtgtccactcgggagatgccactctgatgctgcccacacaaaccatcagccaaggggccattgaaaaggtgaaggatgctacccggaagattgcaaaggcttttgccatctctggtccattcaacgtccaatttcttgtcaaaggaaatgatgtcttggtgattgagtgtaacttgagagcttctcgatccttcccctttgtttccaagactcttggggttgacttcattgatgtggccaccaaggtgatgattggagagaatgttgatgagaaacatcttccaacattggaccatcccataattcctgctgactatgttgcaattaaggctcccatgttttcctggccccggttgagggatgctgaccccattctgagatgtgagatggcttccactggagaggtggcttgctttggtgaaggtattcatacagccttcctaaaggcaatgctttccacaggatttaagataccccagaaaggcatcctgataggcatccagcaatcattccggccaagattccttggtgtggctgaacaattacacaatgaaggtttcaagctgtttgccacggaagccacatcagactggctcaacgccaacaatgtccctgccaccccagtggcatggccgtctcaagaaggacagaatcccagcctctcttccatcagaaaattgattagagatggcagcattgacctagtgattaaccttcccaacaacaacactaaatttgtccatgataattatgtgattcggaggacagctgttgatagtggaatccctctcctcactaattttcaggtgaccaaactttttgctgaagctgtgcagaaatctcgcaaggtggactccaagagtcttttccactacaggcagtacagtgctggaaaagcagcatagagatgcagacaccccagccccattattaaatcaacctgagccacatgttatctaaaggaactgattcacaactttctcagagatgaatattgataactaaacttcatttcagtttactttgttatgccttaatattctgtgtcttttgcaattaaattgtcagtcacttcttcaaaaccttacagtccttcctaagttactcttcatgagatttcatccatttactaatactgtatttttggtggactaggcttgcctatgtgcttatgtgtagctttttactttttatggtgctgattaatggtgatcaaggtaggaaaagttgctgttctattttctgaactctttctatactttaagatactctatttttaaaacactatctgcaaactcaggacactttaacagggcagaatactctaaaaacttgataaaattaaatatagatttaatttatgaaccttccatcatgatgtttgtgtattgcttctttttggatcctcattctcacccatttggctaatccaggaatattgttatcccttcccattatattgaagttgagaaatgtgacagaggcatttagagtatggacttttcttttctttttctttttctttttttctttttgagatggagtcacactctccaggctggagtgcagtggcacaatctcggctcactgcaatttccgtctcccaagttcaagcgattctcctgctttagactatggatttctttaaggaatactggtttgcagttttgttttctggactatatcagcagatggtagacagtgtttatgtagatgtgttgttgtttttatcattggattttaacttggcccgagtgaaataatcagatttttgtcattcacactctcccccagttttggaataacttggaagtaaggttcattcccttaagacgatggattctgttgaactatggggtcccacactgcactattaattccacccactgtaagggcaaggacaccattccttctacatataagaaaaaagtctctccccaagggcagcctttgttacttttaaatattttctgttattacaagtgctctaattgtgaacttttaaataaaatactattaagaggtaa

SEQ ID NO: 2 - protein, 1500 aa, Homo sapiens
MTRILTAFKVVRTLKTGFGFTNVTAHQKWKFSRPGIRLLSVKAQTAHIVLEDGTKMKGYSFGHPSSVAGEVVFNTGLGGYPEAITDPAYKGQILTMANPIIGNGGAPDTTALDELGLSKYLESNGIKVSGLLVLDYSKDYNHWLATKSLGQWLQEEKVPAIYGVDTRMLTKIIRDKGTMLGKIEFEGQPVDFVDPNKQNLIAEVSTKDVKVYGKGNPTKVVAVDCGIKNNVIRLLVKRGAEVHLVPWNHDFTKMEYDGILIAGGPGNPALAEPLIQNVRKILESDRKEPLFGISTGNLITGLAAGAKTYKMSMANRGQNQPVLNITNKQAFITAQNHGYALDNTLPAGWKPLFVNVNDQTNEGIMHESKPFFAVQFHPEVTPGPIDTEYLFDSFFSLIKKGKATTITSVLPKPALVASRVEVSKVLILGSGGLSIGQAGEFDYSGSQAVKAMKEENVKTVLMNPNIASVQTNEVGLKQADTVYFLPITPQFVTEVIKAEQPDGLILGMGGQTALNCGVELFKRGVLKEYGVKVLGTSVESIMATEDRQLFSDKLNEINEKIAPSFAVESIEDALKAADTIGYPVMIRSAYALGGLGSGICPNRETLMDLSTKAFAMTNQILVEKSVTGWKEIEYEVVRDADDNCVTVCNMENVDAMGVHTGDSVVVAPAQTLSNAEFQMLRRTSINVVRHLGIVGECNIQFALHPTSMEYCIIEVNARLSRSSALASKATGYPLAFIAAKIALGIPLPEIKNVVSGKTSACFEPSLDYMVTKIPRWDLDRFHGTSSRIGSSMKSVGEVMAIGRTFEESFQKALRMCHPSIEGFTPRLPMNKEWPSNLDLRKELSEPSSTRIYAIAKAIDDNMSLDEIEKLTYIDKWFLYKMRDILNMEKTLKGLNSESMTEETLKRAKEIGFSDKQISKCLGLTEAQTRELRLKKNIHPWVKQIDTLAAEYPSVTNYLYVTYNGQEHDVNFDDHGMMVLGCGPYHIGSSVEFDWCAVSSIRTLRQLGKKTVVVNCNPETVSTDFDECDKLYFEELSLERILDIYHQEACGGCIISVGGQIPNNLAVPLYKNGVKIMGTSPLQIDRAEDRSIFSAVLDELKVAQAPWKAVNTLNEALEFAKSVDYPCLLRPSYVLSGSAMNVVFSEDEMKKFLEEATRVSQEHPVVLTKFVEGAREVEMDAVGKDGRVISHAISEHVEDAGVHSGDATLMLPTQTISQGAIEKVKDATRKIAKAFAISGPFNVQFLVKGNDVLVIECNLRASRSFPFVSKTLGVDFIDVATKVMIGENVDEKHLPTLDHPIIPADYVAIKAPMFSWPRLRDADPILRCEMASTGEVACFGEGIHTAFLKAMLSTGFKIPQKGILIGIQQSFRPRFLGVAEQLHNEGFKLFATEATSDWLNANNVPATPVAWPSQEGQNPSLSSIRKLIRDGSIDLVINLPNNNTKFVHDNYVIRRTAVDSGIPLLTNFQVTKLFAEAVQKSRKVDSKSLFHYRQYSAGKAA

(19) United States
(12) Patent Application Publication
(10) Pub. No.: US 2023/0236204 A1
(43) Pub. Date: Jul. 27, 2023

(54) NON INVASIVE METHODS FOR DIAGNOSING LIVER FIBROSIS

(71) Applicant: King's College Hospital NHS Foundation Trust, London (GB)

(72) Inventor: Saima AJAZ, London (GB)

(21) Appl. No.: 17/907,162

(22) PCT Filed: Mar. 24, 2021
(86) PCT No.: PCT/GB2021/050714

§ 371 Date: Sep. 23, 2022

(30) Foreign Application Priority Data
Mar. 27, 2020 (GB) 2004471. 5

(51) Int. Cl. G01N 33/68; G01N 33/543

(52) U.S. Cl. CPC G01N 33/6893; G01N 33/6812; G01N 33/54386; G01N 2333/9015; G01N 2800/085

(57) ABSTRACT

The invention relates to a method comprising a) providing a blood sample from a subject b) determining the level of CPS-₁ expression in said sample c) comparing the level of CPS-₁ expression of (b) to the level of CPS-₁ expression determined from a blood sample from a subject with mild to moderate fibrosis of the liver d) determining the level of glutamate in said sample e) comparing the level of glutamate of (d) to level of glutamate determined from a blood sample from a subject with mild to moderate fibrosis of the liver f) wherein if the level of glutamate of (d) and CPS-₁ expression of (b) is higher than the level of glutamate and CPS-₁ expression from a blood sample from a subject with mild to moderate fibrosis of the liver, it is inferred the subject has increased likelihood of having advanced or severe (F3/F4) fibrosis of the liver.

### FIELD OF THE INVENTION

The invention is in the field of liver function, particularly liver fibrosis and its diagnosis and staging, more particularly in patients with Non-Alcoholic Fatty Liver Disease.

### BACKGROUND

Non-Alcoholic Fatty Liver Disease (NAFLD) is the fastest growing epidemic due to obesity and metabolic syndrome. Currently there are no FDA approved drugs for treatment of NAFLD. Liver biopsy is the “gold standard” diagnostic test for Non-Alcoholic Steatohepatitis (NASH). There is an urgent need for identification of non-invasive biomarkers not only for early detection of patients with NASH, but which could also predict the progression of fibrosis with the disease. Imaging techniques and blood biomarkers are being evaluated extensively but most of them are unable to differentiate different stages of fibrosis, and some are difficult to utilise because of high cost.

In the art, biopsy is considered the ‘gold standard’ for determining the degree of fibrosis in NAFLD patients. It is invasive, qualitative and associated with risks, which are all drawbacks in the art. Moreover, it is costly as well as being difficult to repeat. NAFLD does not affect liver uniformly and hence sampling error is common. Poor quality of sample and analysis bias are further disadvantages associated with biopsy. These are serious problems in the art.

Imaging techniques such as Transient elastography (TE), ultrasound scan (USS/sonogram) and magnetic resonance imaging (MRI) are also used for diagnosing disease progression in NAFLD. TE and USS are both operator dependent. USS cannot differentiate degree of fibrosis. MRI, although highly sensitive and specific, is very costly. In addition, MRI may require expensive contrast agents to be injected as well as extended periods in the claustrophobic environment of the scanner, both of which can produce patient compliance problems as well as requiring highly trained staff to administer. These are problems in the art.

Non-invasive markers to date have limited accuracy for individual patients. (Wong et al 2018 (Wong, V.W., Adams, L.A., de Lédinghen, V. et al. Noninvasive biomarkers in NAFLD and NASH ---- current progress and future promise. Nat Rev Gastroenterol Hepatol 15, 461---478) reviews current and potential biomarkers (including blood biomarkers) for different features of NAFLD, namely steatosis, necroinflammation and fibrosis. For each biomarker, Wong et al evaluate its accuracy, reproducibility, responsiveness, feasibility and limitations. Wong et al summarise on page 467, right column “Summary and Recommendations”: “CK18 is the most extensively evaluated test for NASH diagnosis, but overall accuracy is moderate at best. Although other biomarkers or panels might hold promise, most have not been independently validated. At present, none of the NASH biomarkers are ready for routine clinical use.”

Moreover, this paper is concerned with NAFLD progression. This paper is not concerned with fibrosis. Thus, there remain no reliable/accurate biomarkers, which is a problem in the art.

There is a computational tool called ‘NAFLD simulator’ available for prediction of mortality. This is made available by Harvard University (MGH Inst. for Tech. Assessment, Harvard Medical School, 101 Merrimac St. STE 1010, Boston, MA 02114, United States - https://mgh-ita-calculators.shinyapps.io/nafld-simulator/). It is an interactive, open-access tool for the short- and long-term risks associated with NAFLD and NASH. However, the degree of fibrosis has to be supplied for the tool to calculate the risk of complications associated with NAFLD. No aspect of the tool is able to diagnose or predict the degree of fibrosis - this value has to be externally supplied for the tool to function. In addition, it is important to emphasise that these are predictive markers for mortality. This tool is not concerned with the assessment nor prediction nor diagnosis of liver fibrosis. Indeed, if a person skilled in the art attempts to use this tool, they find that fibrosis data has to be provided and is in no way an output or inference from the use of this tool. This is a problem in the art.

WO2018/037229 discloses a method of diagnosing, prognosing or monitoring or staging the progression of non-alcoholic fatty liver disease (NAFLD) in an individual, the method comprising detecting and quantifying one or more biomarkers in a biological sample obtained from the individual, wherein the one or more biomarkers is selected from apolipoprotein F, lipopolysaccharide-binding protein, ficolin-2, apolipoprotein D, kininogen-1, apolipoprotein M, thrombospondin-1, IgG Fc-binding protein, cystatin-c, alpha- 1 -acid glycoprotein 2, and leucine-rich alpha-2-glycoprotein, and thereby, diagnosing, prognosing or monitoring or staging the progression of NAFLD. The invention does not relate to any of these biomarkers.

DeChiara et al 2018 (J.Hepatol. 2018 vol 69 pages 905-915) discloses urea cycle dysregulation in non-alcoholic fatty liver disease. The authors disclose that NASH is associated with a reduction in the gene and protein expression, and activity, of urea cycle enzymes (UCEs). This results in hyperammonemia, possibly through hypermethylation of UCE genes and impairment of urea synthesis. This document proposes a link between NASH, the function of UCEs, and hyperammonemia. This document proposes ammonia as a potential target for prevention of progression of NASH. It is important to note that, even if the theories advanced in this document are correct, hyperammonemia is proposed to precede, and may or may not be causative for, scarring/fibrosis. This document does not disclose detection/diagnosis of fibrosis.

Chacko et al 2019 (Redox Biology vol 22 doc i.d. 101165) disclose a study of mitochondria in precision medicine; linking bioenergetics and metabolomics in platelets. The document concludes that the metabolome in the intact platelet is functionally integrated with bioenergetics. It is suggested that platelets energetics and metabolomics could be used to assess the susceptibility of a population to environmental exposure and the severity of the toxic response among individuals, and possible relationship to Alzheimer’s and other age-related pathologies. There is no mention of liver fibrosis.

There is no non invasive test for liver fibrosis known in the art. Currently the only tests which can give indications about fibrosis are invasive, involving biopsy, or at the very least involving scanning/elasticity testing/magnetic resonance imaging (MRI) techniques. The current clinical “gold standard” is liver biopsy. This is an invasive procedure which carries with it significant clinical risks. Indeed, the current clinical guidelines clearly state that a biopsy should not be used unless the patient/subject is suspected of having advanced severe fibrosis (i.e. F3/F4 fibrosis of the liver). These are serious problems in the art.

The present invention seeks to overcome problem(s) associated with the prior art.

### SUMMARY

The inventors teach a new non invasive method for assessing fibrosis (i.e. liver fibrosis). The non invasive method is advantageously carried out on a sample such as a blood sample from the subject. The new method involves a very small number of biomarkers which have been carefully selected to provide extremely high statistical confidence in both specificity and sensitivity of the assay.

The inventors arrived at the invention after an extended and intellectually demanding process involving numerous surprising insights. Firstly, the inventors examined untargeted metabolomics data which initially involved more than 490 metabolites being analysed. In parallel to this, the inventors carried out a bioenergetic study on blood, something which has never been applied in the field of liver function before. As a result of several instances of inventive inspiration which are explained in more detail below, the inventors then made a dramatic insight into the underlying biochemistry exhibited by patients with advanced/severe fibrosis and took the ground breaking decision to combine analysis of particular enzymes selected from the citric acid or urea cycle together with selected metabolites present in the blood. Taking further innovative decisions in order to minimise the number of markers and select the best performing combination, the inventors discarded various markers and produced the very small and robust selection (“panel”) of markers which demonstrate extremely robust statistical performance in diagnosing /detecting severe or advanced fibrosis of the liver.

The invention is based on these surprising findings.

Thus in one aspect the invention relates to a method comprising
a) providing a blood sample from a subject
b) determining the level of CPS-1 expression in said sample
c) comparing the level of CPS-1 expression of (b) to the level of CPS-1 expression determined from a blood sample from a subject with mild to moderate fibrosis of the liver
d) determining the level of glutamate in said sample
e) comparing the level of glutamate of (d) to the level of glutamate determined from a blood sample from a subject with mild to moderate fibrosis of the liver
f) wherein if the level of CPS-1 expression of (b) is higher than the level of CPS-1 expression determined from a blood sample from a subject with mild to moderate fibrosis of the liver, and
   the level of glutamate of (d) is higher than the level of glutamate determined from a blood sample from a subject with mild to moderate fibrosis of the liver,
   then it is inferred that the subject has an increased likelihood of having advanced or severe (F3/F4) fibrosis of the liver.

In another embodiment the invention relates to a method comprising
a) providing a blood sample from a subject
b) determining the level of arginine in said sample
c) comparing the level of arginine of (b) to the level of arginine determined from a blood sample from a subject with mild to moderate fibrosis of the liver
d) determining the citrulline/ornithine ratio in said sample
e) comparing the citrulline/ornithine ratio of (d) to the citrulline/ornithine ratio determined from a blood sample from a subject with mild to moderate fibrosis of the liver
f) determining the reserve capacity in said sample
g) comparing the reserve capacity of (f) to the reserve capacity determined from a blood sample from a subject with mild to moderate fibrosis of the liver
h) wherein if the level of arginine of (b) is lower than the level of arginine determined from a blood sample from a subject with mild to moderate fibrosis of the liver, and the citrulline/ornithine ratio of (d) is lower than the citrulline/ornithine ratio determined from a blood sample from a subject with mild to moderate fibrosis of the liver, and
   the reserve capacity of (f) is lower than the reserve capacity determined from a blood sample from a subject with mild to moderate fibrosis of the liver,
   then it is inferred that the subject has an increased likelihood of having advanced or severe (F3/F4) fibrosis of the liver.

Suitably the CPS-1 level determined is the plasma CPS-1 level.

Suitably the CPS-1 level is determined by quantitative sandwich immunoassay. Suitably said quantitative sandwich immunoassay comprises an ELISA assay.

Suitably the level of glutamate is determined using mass spectrometry.

Suitably the level of arginine is determined using mass spectrometry.

Suitably the levels of citrulline/ornithine are determined using mass spectrometry.

Suitably the reserve capacity is determined as maximal OCR minus basal respiration.

Suitably the reserve capacity is determined using the ‘XF cell mito stress test kit’ from Agilent Technologies.

Suitably the subject is suspected of having liver fibrosis.

Suitably the subject has been previously identified as having F0-F2 liver fibrosis, preferably F1-F2 liver fibrosis.

Suitably the subject is suspected of having, or has, metabolic syndrome.

Suitably the subject is suspected of having, or has, diabetes, preferably type 2 diabetes.

Suitably the subject is suspected of having, or has, Non-Alcoholic Fatty Liver Disease (NAFLD).

Suitably the subject is suspected of having, or has, Non-Alcoholic Steatohepatitis (NASH).

In another embodiment the invention relates to a method comprising
a) providing a first blood sample from a subject taken at a first time point;
b) either
   i. determining the level of CPS-1 expression in said sample and determining the level of glutamate in said sample, or
   ii. determining the level of arginine in said sample, determining the citrulline/ornithine ratio in said sample and determining the reserve capacity in said sample;
c) providing a second blood sample from a subject taken at a second time point;
d) determining the same characteristics as were determined in step (b) for said second blood sample of step (c)
e) comparing the values from step (b) to the values from step (d);
f) inferring from the comparison of step (e) whether fibrosis has changed wherein if the values from step (b) and step (d) are different, then it is inferred that fibrosis has changed in the subject.

Suitably if the level of CPS-1 expression in said second sample and the level of glutamate in said second sample are higher than the levels for said first sample, then it is inferred that fibrosis has advanced or increased in said patient, and wherein if the level of CPS-1 expression in said second sample and the level of glutamate in said second sample are lower than the levels for said first sample, then it is inferred that fibrosis has receded or decreased in said patient.

Suitably if the level of arginine in said second sample, the citrulline/ornithine ratio in said second sample and the reserve capacity in said second sample are lower than the levels for said first sample, then it is inferred that fibrosis has advanced or increased in said patient, and wherein if the level of arginine in said second sample, the citrulline/ornithine ratio in said second sample and the reserve capacity in said second sample are higher than the levels for said first sample, then it is inferred that fibrosis has receded or decreased in said patient.

Suitably the method is a method of monitoring a subject’s progress over time

Suitably the method is a method of assessing changes in fibrosis over time

Suitably the method is a method of determining response to treatment. Treatment may be pharmaceutical e.g. administration of one or more active compound(s), or treatment may be behavioural e.g. diet, exercise regime, or other factor.

In another embodiment the invention relates to a method of treating a subject with fibrosis of the liver, the method comprising performing a method as described above and if it is inferred that the subject has an increased likelihood of having advanced or severe (F3/F4) fibrosis of the liver, then one or more treatments selected from the group consisting of: reformed diet, exercise regime, low calorie diet, and administration of GLP analogue, such as a weekly injection of GLP analogue, is administered to said subject.

### DETAILED DESCRIPTION

The inventors’ unique approach for biomarkers is based on bioenergetics and metabolomics and can detect mechanistic abnormalities in disease pathogenesis of NAFLD. This is the first study to date in which a combined approach using real-time energy changes in peripheral blood immune cells and the corresponding metabolite changes using untargeted global metabolomics has been used to investigate disease progression in NAFLD.

This also helps to shed light on the molecular mechanisms underlying progression of disease.

“Invasive” has its normal meaning in the art. Collecting a sample of biological fluid such as a blood sample is not considered invasive.

“Level” means concentration, for example expressed as ng/ml e.g. ng substance of interest/ml sample. Suitably the sample is plasma. For consistency this may be adjusted to represent ng substance/ml blood if desired. The important point is that when comparing values they are all either per ml plasma (or per ml sample or per ml blood etc) - the absolute meaning is not important provided that the units are the same between the figures being compared according to usual scientific practice.

It will be noted that the ‘citrulline/ornithine ratio’ does not have units because one concentration divided by another concentration leaves an integer (i.e. the ratio) which does not have a unit.

The inventors teach that advanced fibrosis is associated with mitochondrial dysfunction in peripheral immune cells in patients with Non-Alcoholic fatty liver disease (NAFLD).

Non-Alcoholic Fatty Liver Disease (NAFLD) is the fastest growing epidemic due to obesity and metabolic syndrome. The global prevalence of NAFLD is 24% (Younossi, et al) and it is expected that Non-Alcoholic Steatohepatitis (NASH) will increase by 63% between 2015 and 2030 driven by rising levels of obesity. The incidence of decompensated cirrhosis due to NAFLD is expected to increase 168% by 2030, while incidence of HCC will increase by 137% (Estes, C. et al). It is believed that the actual prevalence of NAFLD and NASH is higher than estimated previously. In patients with type 2 diabetes the prevalence is estimated to be 70% (Mantovani A,et al). NAFLD is regarded as the hepatic manifestation of the metabolic syndrome (Marchesini G, et al).

The spectrum of NAFLD ranges from accumulation of simple fat around the liver also known as steatosis to inflammatory phase defined as NASH. Subsequently, this process can lead to scarring to the liver and can progress to cirrhosis, and even hepatocellular carcinoma (HCC).

The mechanisms involved in the progression of benign fatty accumulation in the liver to inflammation and ultimately to scar tissue or cirrhosis are incompletely understood. Dysfunctional metabolism lies at the centre of NASH/NAFLD and includes defects in mitochondrial function, lipid metabolism, cholesterol metabolism and inflammation (Bellanti et al). Insulin resistance and obesity which are hallmarks of NAFLD are characterised with imbalance of energy expenditure. This can be investigated via measuring real time energy changes in peripheral cells and metabolomic screening. Mitochondria are the main energy source in hepatocytes and play a major role in extensive oxidative metabolism and normal function of the liver. Using new assays which have been developed in the last few years its possible to measure cellular bioenergetics and mitochondrial dysfunction. Numerous studies have proposed that peripheral blood immune cells can act as sensors of mitochondrial dysfunction in chronic diseases (Rudkowska, Raymond et a, Czajka et al, Maynard, S.et al, Hartman et al). The peripheral blood may reflect the systemic changes in the disease and provide a valuable tool for clinical studies due to ease of availability and storage. The oxidative stress is considered a potential initiating factor that results in the inflammatory reactions involved in the occurrence of fibrosis (Yeh et al., 2007). It is also linked to increased production of cytokine in various metabolic diseases (Incalza et al., 2018) The inventors propose that defects in mitochondrial function or bioenergetics deficit in immune cells can be utilised as a predictive marker for progression of fibrosis in NASH. Mitochondrial dysfunction and associated oxidative stress can have functionally important immune consequences such as increased production of pro-inflammatory cytokines. This causes a vicious cycle which promotes the inflammation, fibrosis and necrosis associated with NASH. These metabolic abnormalities can be detected in peripheral cells and can be used as non-invasive biomarkers of liver fibrosis in NASH progression reflecting the hepatic changes.

Metabolomics is the large-scale study of small molecules like glucose or cholesterol produced by cellular activity. Use of metabolomics is widely used for quantifying range of molecular intermediates from major bioenergetics pathways. It can also explain the mechanisms involved in the pathogenesis of disease and highlight therapeutic targets (Gowda et al; Nicholas J et al; Delzenne and Bindels). Integrative analysis of untargeted metabolomic which can measure metabolite masses and predict the identity of each metabolite paired with bioenergetics and clinical information can help to detect mechanistic abnormalities in disease pathogenesis of complex diseases such as NAFLD. This is the first study to date in which a combined approach using real-time energy changes in peripheral blood immune cells and the corresponding metabolite changes using untargeted global metabolomics is used to investigate disease progression in NAFLD.

The inventors investigated bioenergetics and the metabolomics from biofluids of patients with fatty liver diseases and have based the invention on their insights. This also helps to shed light on the molecular mechanisms leading to progression of disease. This understanding can be used to for development of non-invasive biomarkers for fibrosis in NASH.

### Biomarker Selection

It is an advantage of the invention that subjects with severe fibrosis can be identified.

The view in the art is that metabolic syndrome has very many pathways involved and is tied up with liver fibrosis in complex ways which are not understood in the art. The inventors took the decision to try to identify biomarkers connected with fibrosis, and to remove overlapping complications with metabolic syndrome.

One approach selected by the inventors was to undertake global untargeted metabolomics. This is a technically demanding procedure which is a substantial deterrent for the person skilled in the art. It should be noted that the tiny number of metabolites which the inventors teach to focus on in the biomarker panels of the invention can be easily assayed now that they have been identified. However, from the start position at the earliest priority date, i.e. without the benefit of hindsight and without knowledge of the invention, the identity of those metabolites was completely unknown and undertaking an untargeted metabolomics analysis in order to try to approach this problem would not have been considered by the skilled person and furthermore represented an unappealing route and a significant barrier for workers in the field.

The inventors collected data from more than 490 metabolites. This was reduced to 13 metabolites and further reduced upon further intellectual decisions taken by the inventors to only 5 candidate metabolites. Unusually, the inventors brought a large background of knowledge from biochemistry and energetics to the unrelated field of liver fibrosis. This enabled the inventors to make intellectual insights such as pinpointing some of the candidate metabolites as being involved with mitochondrial dysfunction.

In parallel, the inventors took the unusual decision to carry out bioenergetic studies on blood. During the complex analysis of these results, the inventors were again able to draw on their unconventional background and training in a completely different field of research to the field of liver fibrosis, and had the intellectual insight that the bioenergetics data seemed also to point to a possible mitochondrial dysfunction.

As a result of researching this topic further, the inventors established that the key 5 metabolites were all part of the citric acid or urea cycle, which they realised only happens within mitochondria. In addition, the inventors had the further insight that the CPS1 enzyme is likely the only single enzyme shared between both the citric acid and the urea cycles. The inventors established that this enzyme is only expressed in liver hepatocytes, and within those cells is only expressed in mitochondria.

This series of insights combined a deep knowledge and original thought from at least two different fields and therefore represents an advance beyond what might be expected from a person of ordinary skill in the art.

In order to further illustrate the complexity of arriving at the present invention, it should be noted that the inventors undertook to check the whole of the urea cycle, reusing their untargeted metabolomics data. When the inventors were able to confirm that they did not see other metabolite changes amongst those enormous range of molecules which they had studied, this further confirmed their dramatic proposition of the biomarker panels which could be used to infer advanced or severe liver fibrosis.

### ‘Increased’ Panel

The inventors teach that the minimised/empirical panel of markers involved assaying only glutamate and CPS1. Data is provided in support of these statistical significance of this panel of biomarkers.

### ‘Decreased’ Panel

The inventors teach assay of three metabolites plus an energetic factor. More specifically, the inventors teach the assay of arginine levels plus the citrulline/ornithine ratio, together with “reserve capacity”.

The inventors selected reserve capacity as the most significant bioenergetic marker. A range of intellectual insights led them to this decision, most importantly because this is taught by the inventors to be the most significant bioenergetic marker due to combining several underlying energetic factors together in a single numeric value.

It should be emphasised that all of the markers separately showed significant differences between mild/moderate fibrosis and advanced/severe fibrosis. Thus, a “standard” approach might have been to use single biomarkers, or might have been to combine all of the biomarkers examined into a single expanded panel following the general principle that more data points can lead to more reliable results. However, instead the inventors went against conventional thinking in the art and made particular combinations of markers and specifically chose to drop certain markers from the analysis whilst examining the effect on the receiver operating characteristic (ROC) curves in order to thoughtfully raise the specificity and/or sensitivity of their methods, which is a further indicator towards inventive step.

The inventors disclose panel(s) of non-invasive biomarkers for Non-Alcoholic Fatty Liver Disease (NAFLD), in particular for assessment of liver fibrosis in NAFLD and/or assessment of liver fibrosis in other settings.

Each panel of biomarkers can be incorporated as a kit to detect and measure the biomarkers in a biological sample from patient(s) e.g. patient(s) with NAFLD.

The inventor has created a unique panel of non-invasive biomarkers for diagnosing and staging the progression of fibrosis in patients, most suitably patients with NAFLD, based on blood biomarkers. These biomarker panels are over or under expressed and the panels have utility as a test for disease progression, most suitably disease progression in NAFLD.

### NASH/NAFLD AND LIVER INFLAMMATION

The mechanisms involved in the progression of benign fatty accumulation in the liver to inflammation and ultimately to scar tissue or cirrhosis are incompletely understood. Dysfunctional metabolism lies at the centre of NASH/NAFLD and includes defects in mitochondrial function, lipid metabolism, cholesterol metabolism and inflammation. Insulin resistance and obesity which are hallmarks of NAFLD are characterised with imbalance of energy expenditure. This can be investigated via measuring real time energy changes in peripheral cells and metabolomic screening.

The peripheral blood may reflect the systemic changes in the disease and provide a valuable tool for clinical studies due to ease of availability and storage. Oxidative stress is considered a potential initiating factor that results in the inflammatory reactions involved in the occurrence of fibrosis.

In the current study we propose that defects in mitochondrial function or bioenergetics deficit in immune cells can be utilised as a predictive marker for progression of fibrosis in NASH. Mitochondrial dysfunction and associated oxidative stress can have functionally important immune consequences such as increased production of pro-inflammatory cytokines. This causes a vicious cycle which promotes the inflammation, fibrosis and necrosis associated with NASH.

Use of metabolomics is widely used for quantifying range of molecular intermediates from major bioenergetics pathways.

The inventors used integrative analysis of untargeted metabolomics which can measure metabolite masses and predict the identity of each metabolite paired with bioenergetics and clinical information to help to detect mechanistic abnormalities in NAFLD.

The inventor shows that metabolic abnormalities can be detected in peripheral blood cells and can be used as a panel of non-invasive biomarkers of liver fibrosis, for example in NASH progression, reflecting the hepatic changes.

### Classification of Fibrosis

Hepatic fibrosis or liver fibrosis (often referred to simply as ‘fibrosis’ herein) is reported using a scale ranging from F0-F4.

In more detail, liver fibrosis is a change in the microscopic structure of the liver, usually as a result of liver inflammation. Liver fibrosis progresses to cirrhosis. Although a complex set of factors may be involved depending on the subject’s medical condition, in some cases abstaining from alcohol and/or lifestyle changes (diet, exercise) and/or treating other conditions (e.g. hypercholesterolaemia, diabetes) and/or other intervention(s) may stop or delay the fibrosis from further progression into significant or severe fibrosis and/or cirrhosis. The latter lead to complications of underlying diseases, including cancer (e.g. hepatocellular carcinoma (HCC)).

Classification of liver fibrosis is well known to the person skilled in the art. In case any further guidance is required, this is outlined below.

Measurement of the amount of fibrosis is called staging. There are five stages (F0; F1; F2; F3; F4).

| | | |
|---|---|---|
| Fibrosis Scale/Score | Subject Liver Condition | Notes |
| F0 | no fibrosis | no scarring |
| F1 | minimal fibrosis | minimal scarring (mild fibrosis) around liver sinusoids or around portal triad |
| F2 | moderate fibrosis | scarring includes both liver sinusoids and portal triad |
| F3 | advanced fibrosis | fibrosis spreading and forming bridges with other fibrotic liver areas |
| F4 | severe fibrosis | cirrhosis or advanced scarring |

Fibrosis scores may overlap (e.g. a patient may be described as F0/F1, F1/F2).

For subjects with F0 fibrosis, they have no fibrosis.

For subjects with F1 to F2 fibrosis, they have either mild or moderate fibrosis.

For subjects having F3 or F4 fibrosis, they have advanced or severe fibrosis. These subjects also have chances of cirrhosis. These subjects, especially F4 subjects, have enhanced chances of hepatocellular carcinoma (HCC).

For the methods of the invention, comparisons of the subject of interest’s scores should most suitably be made to F1/F2 values. Thus suitably comparisons of the subject of interest’s scores are not made to F0 values.

By way of explanation, it will be noted that F0 is not necessarily a healthy individual. The NASH clinical research network (NASH CRN) histological scoring has 2 components: NAS score (a) Steatosis (0, 1, 2 and 3) (b) Lobular inflammation (0, 1, 2 and 3) (c) Hepatocellular ballooning (0, 1 and 2); and Fibrosis score : 0, 1, 2, 3 and 4. So even if fibrosis is 0 this does not indicate healthy patient because the patient can have a varying NAS score which shows fat presence and inflammatory phases of NAFLD. The disease NAFLD broadly has 4 stages (1) simple fatty liver (2) fatty liver with hepatocyte inflammation (NASH) (3) mild fibrosis to moderate fibrosis (4) advanced fibrosis. So fibrosis can be zero but fatty liver and inflammation can be present which are early stages of NAFLD. For the avoidance of doubt, the invention is focussed on assessing fibrosis/likelihood of fibrosis/advance or increase of fibrosis/receding or decrease of fibrosis in a subject. The invention may find application in constructing a NASH CRN histological score, but the invention is focussed on only the fibrosis part of that score. The examples section provides extensive data, which data comprises studies based on NAFLD patients and biomarkers for fibrosis development. There are healthy controls as positive controls but suitably the methods of the invention involve comparison to F1/F2 control values as stated in the appended claims. Suitably the methods of the invention are not based on changes/comparisons to healthy controls.

### Detection

Any suitable method for measuring proteins such as CPS-1 can be used such as spectrometry, absorbance, protein immunostaining, western blot, dot blot and/or ELISA.

Suitably the metabolites described herein (for example glutamate, arginine, citrulline, ornithine etc.) are detected by any suitable means known in the art. Suitably mass spectrometry is used. Most suitably quantitative mass spectrometry is used.

The invention is not tied to a particular brand or supplier of mass spectrometry instruments or services.

The particular type of mass spectrometry may be selected by the skilled operator depending on their needs.

It may be desirable to select the mass spectrometry from the following types:
1) Gas chromatography-mass spectrometry (GC-MS);
2) Liquid chromatography-mass spectrometry (LC-MS)

This choice is typically determined by the volatility of the compounds of interest as to which mobile phase (i.e. the liquid or the gas) is more suitable for the analysis.

For greater specificity, and/or to analyse higher molecular weight molecules, it may be desirable to use MS/MS (tandem mass spectrometry, occasionally referred to as MS²). This involves a first MS separation by mass to charge ration (m/z ratio), followed by fragmentation of the peaks of interest and analysis of those fragments by second MS (hence MS-MS). This is also a standard technique well known in the art. Of course the techniques may be coupled together (LC-MS, LC-MSMS, GC-MS, GC-MSMS etc.) according to operator needs.

In one embodiment the MxP® Global Profiling system is used to detect and/or quantify metabolite levels. This is commercially available from Metalomics Health i.e. from BIOCRATES Life Sciences AG, Eduard-Bodem-Gasse 8 6020 Innsbruck, Austria.

### Glutamate

Suitably glutamate levels may be assessed by any means known to the person skilled in the art.

Glutamate levels may be determined by mass spectrometry.

Glutamate levels may be determined by LC-MS, LC-MSMS, GC-MS, or GC-MSMS.

Suitably glutamate levels are assessed using the MxP® Global Profiling system. This is commercially available from Metalomics Health i.e. from BIOCRATES Life Sciences AG, Eduard-Bodem-Gasse 8 6020 Innsbruck, Austria.

Unless otherwise apparent from the text, suitably the level of glutamate refers to the plasma level of glutamate.

### Detection/Masses/Peaks

The skilled person can identify the metabolites mentioned herein, which are chemically well known. In case any further guidance is needed, we refer to table A below.

TABLE A

details of biomarkers useful in the invention

| Metabolite | Molecular Weight And Official Compound Identifier (‘PubChem CID’) | Cohort Value (Quantitative [µmol/L] F1-F2 Fibrosis | Cohort Value (Quantitative [µmol/L] F3-F4 Fibrosis | Notes |
|---|---|---|---|---|
| Glutamate | 147.13 g/mol PubChem CID: 23327 | 64 | 137 | The Mass spec data was quantified using MxP® Global Profiling QUANT platform and metabolite were quantified as µmol/L. |
| Arginine | 174.2 g/mol PubChem CID: 6322 | 62 | 40 | The Mass spec data was quantified using MxP® Global Profiling QUANT platform and metabolite were quantified as µmol/L. |
| Citrulline | 175.19 g/mol PubChem CID: 9750 | 24 | 20 | The Mass spec data was quantified using MxP® Global Profiling QUANT platform and metabolite were quantified as µmol/L. |
| Ornithine | 132.16 g/mol PubChem CID: 6262 | 61 | 89 | The Mass spec data was quantified using MxP® Global Profiling QUANT platform and metabolite were quantified as µmol/L. |

### Reference Sequences

Suitably all sequences herein are discussed with reference to human CPS-₁ provided as SEQ ID NO: 1 (nucleic acid) and SEQ ID NO: 2 (protein) - see below.

When particular amino acid residues are referred to herein using numeric addresses, the numbering is taken with reference to the human CPS-₁ amino acid sequence (or to the polynucleotide sequence encoding same if referring to nucleic acid).

This is to be used as is well understood in the art to locate the residue of interest. This is not always a strict counting exercise - attention must be paid to the context. For example, if the protein of interest is of a slightly different length, then location of the correct residue in that sequence may require the sequences to be aligned and the equivalent or corresponding residue picked. This is well within the ambit of the skilled reader.

Mutating has it normal meaning in the art and may refer to the substitution or truncation or deletion of one or more residues, motifs or domains. Mutation may be effected at the polypeptide level, for example, by synthesis of a polypeptide having the mutated sequence, or may be effected at the nucleotide level, for example, by making a polynucleotide encoding the mutated sequence, which polynucleotide may be subsequently translated to produce the mutated polypeptide. Suitably, the mutations to be used are as set out herein. Unless otherwise apparent from the context, mutations mentioned herein are substitutions. For example ‘V10A’ means that the residue corresponding to ‘V10’ in the human CPS-1 amino acid sequence (SEQ ID NO: 2) is substituted with A.

### CPS-1

CPS1 (Carbamoyl Phosphate Synthase-1) levels may be determined by any suitable method known in the art.

Suitably CPS1 means human CPS1. There are several known transcripts for human CPS1, for example in the GenBank database.

Most suitably the reference sequence for CPS1 (Carbamoyl Phosphate Synthase-1) is provided as SEQ ID NO: 1:
Homo sapiens carbamoyl-phosphate synthase 1 (CPSi), transcript variant 2, mRNA 5,760 bp linear mRNA
Accession number (NCBI Reference Sequence): NM_001875, more suitably NM_001875.5

Suitably unless otherwise apparent from the context, ‘CPS1’ means the protein sequence i.e. a polypeptide having the amino acid sequence encoded by the above nucleic acid sequence. For the avoidance of doubt the CPS1 amino acid sequence is provided as SEQ ID NO: 2:

Unless otherwise apparent, accession numbers are for GenBank. GenBank is a sequence database as described in Benson, D. et al, Nucleic Acids Res. 45(D1):D37-D42 (2017). In more detail, GenBank is as administered by the National Center for Biotechnology Information, National Library of Medicine, 38A, 8N805, 8600 Rockville Pike, Bethesda, MD 20894, USA. Suitably the current version of sequence database(s) are relied upon. Alternatively, the release in force at the date of filing is relied upon. For the avoidance of doubt, NCBI-GenBank Release 235 (15 Dec. 2019) is relied upon.

Suitably CPS1 levels means CPS1 polypeptide/ protein levels. Suitably CPS1 levels are determined using a quantitative sandwich immunoassay. Suitably the quantitative sandwich immunoassay comprises an Elisa assay. Most suitably CPS₁ levels are determined using ELISA kit Catalog No: RD-CPS1-Hu from Red Dot Biotech, 201-5309 Main Street, Kelowna, BC, V1W 4V3, Canada. Suitably this is carried out according to the manufacturer’s instructions.

### Arginine

Arginine levels may be determined by any suitable method known in the art.

Arginine levels may be determined by mass spectrometry.

Arginine levels may be determined by LC-MS, LC-MSMS, GC-MS, or GC-MSMS.

Suitably arginine levels are assessed using the MxP® Global Profiling system. This is commercially available from Metalomics Health i.e. from BIOCRATES Life Sciences AG, Eduard-Bodem-Gasse 8 6020 Innsbruck, Austria.

Unless otherwise apparent from the text, suitably the level of arginine refers to the plasma level of arginine.

### Citrulline/Ornithine

Ornithine transcarbamylase (OTC) (also called ornithine carbamoyltransferase) is an enzyme (EC 2.1.3.3) that catalyzes the reaction between carbamoyl phosphate (CP) and ornithine (Orn) to form citrulline (Cit) and phosphate (Pi).

Citrulline/Ornithine levels may be determined by any suitable method known in the art.

Citrulline/Ornithine levels may be determined by mass spectrometry.

Citrulline/Ornithine levels may be determined by LC-MS, LC-MSMS, GC-MS, or GC-MSMS.

Suitably Citrulline/Ornithine levels are assessed using the MxP® Global Profiling system. This is commercially available from Metalomics Health i.e. from BIOCRATES Life Sciences AG, Eduard-Bodem-Gasse 8 6020 Innsbruck, Austria.

Unless otherwise apparent from the text, suitably the level of Citrulline/Ornithine refers to the plasma level of Ornithine.

The Citrulline/Ornithine ratio is calculated using the formula: [Citrulline level]/[Ornithine level]= Citrulline/Ornithine ratio

### Reserve Capacity

Reserve capacity may be determined by any suitable method known in the art.

Suitably reserve capacity may be determined via cellular bioenergetics. Cellular bioenergetics are suitably performed using ‘XF cell mito stress test kit in a Seahorse XFp analyzer (Agilent Technologies).

In one embodiment, PBMCs are extracted from the blood sample and are suspended in XF media and 300,000 cells/well are seeded to Cell-Tak (Beckton Dickinson Ltd) coated XFp plates (Agilent Technologies).

All experiments are suitably performed with 3 replicate wells in the Seahorse XFp analyzer. Oxygen consumption rate (OCR), a measurement of mitochondrial respiration, is measured in the presence of specific mitochondrial activators and inhibitors as required. Oligomycin (ATP synthase blocker) may be used to measure ATP turnover and to determine proton leak. In this embodiment the mitochondrial un-coupler FCCP (carbonyl cyanide 4-[trifluoromethoxy] phenylhydrazone) is used to measure maximum respiratory function (maximal OCR).

Reserve capacity is calculated as maximal OCR minus the basal respiration.

Basal respiration is measured before addition of any activators or inhibitors and is respiration of cells at rest. It is calculated by measuring last measurement before first injection minus non-mitochondrial respiration.

### Medical/Clinical Considerations

It should be noted that approximately 70% of liver fibrosis patients have diabetes, which especially makes the glucose/maltose aspects discussed herein important.

It is an advantage of the invention that it enables fast progressing individuals to be identified.

It is an advantage of the invention that it enables subjects with a high degree of fibrosis to be identified.

The invention finds application in monitoring the progression of fibrosis. This embodiment involves carrying out method invention at different points in time, and comparing the results from those different time points. In this way, the clinician or physician can gain an understanding of whether the patients fibrosis is increasing or decreasing, or remaining unchanged.

In some aspects the invention may be considered as a diagnostic tool, or a tool for collecting information useful in aiding diagnosis.

In some aspects the invention may be considered as a predictive tool, or a tool for collecting information useful in predicting outcomes or progression or resolution, or predicting effect of treatment. Thus in some aspects the test (i.e. the method of the invention) may be repeated for the same subject once a month, once every two months or once every three months or once every six months. The values determined for samples taken at these different time intervals may be compared for the subject. A monthly rate of change can be calculated using the values obtained. These values indicate if the patient’s fibrosis is stable (if values stay the same) or if the patient’s fibrosis is progressing/receding (if values change over time). If it is observed that the values are increasing (for CPS-1 and/or glutamate), or if it is observed that the values are decreasing (for arginine and/or citrulline/ ornithine ratio and/or reserve capacity) in serial tests it can predict if the patients are fast progressors of fibrosis or slow progressors. For example the calculated monthly rate of increase (for CPS-1 and/or glutamate), or the calculated monthly rate of decrease (for arginine and/or citrulline/ ornithine ratio and/or reserve capacity) gives an indication of the rate of progression of fibrosis. Thus the invention can be used to predict progression of different stages of NAFLD.

The reverse of this may be carried out to monitor receding of fibrosis and/or to monitor progression/effectiveness of treatment. For example, if it is observed that the values are decreasing (for CPS-1 and/or glutamate), or if it is observed that the values are increasing (for arginine and/or citrulline/ornithine ratio and/or reserve capacity) in serial tests it can indicate that fibrosis is receding and/or that treatment is effective. For example the calculated monthly rate of decrease (for CPS-1 and/or glutamate), or the calculated monthly rate of increase (for arginine and/or citrulline/ornithine ratio and/or reserve capacity) gives an indication of the rate of recession of fibrosis/an indication of the effectiveness of the treatment or speed of improvement/response to the treatment. Thus the invention can be used to predict recession/resolution/improvement of different stages of NAFLD under treatment. Such monitoring can also help in assessment of effectiveness of a therapy.

The invention finds application as a marker of late stage fibrosis. First the invention finds application in diagnosing the presence of late stage fibrosis (advanced fibrosis/severe fibrosis).

### Subject

Suitably the subject has a liver. Suitably the subject is a vertebrate. Suitably the subject is a mammal. Suitably the subject is a primate. Suitably the subject is a human.

Suitably the subject suspected of having fibrosis.

When the subject is suspected of having fibrosis, the invention finds particular application in the system the clinician or physician to decide whether or not that subject does indeed have fibrosis. In particular, the invention finds application in the system the clinician/physician whether that subject has advanced or severe fibrosis (F3/F4 fibrosis).

It should be noted that the invention provides a breakthrough and that this is the first test for fibrosis of the liver to be described other than invasive biopsy procedure of the prior art. Thus, in a broad aspect the invention provides a biomarker test for liver fibrosis.

Suitably the subject has deranged enzyme levels.

Suitably the subject has low platelets.

Suitably the subject has deranged INR (international normalised ratio - indication of clotting time).

### Combinations

The invention may assist in differentiation between stages of liver fibrosis.

It may be an advantage to combine the invention with additional clinical markers.

For example, the method of the invention could be carried out together with an assessment of platelet levels in the subject. This might facilitate the stratification of patients.

The main use of the invention is as a biomarker to test to indicate presence (or increase likelihood of presence) of advanced fibrosis of the liver.

The invention finds application in identification of subjects with F3/F4 fibrosis.

### Clinical Approaches

Patients with F3/F4 fibrosis are in a severe clinical condition and they may need a transplant or other intervention.

It should be noted that histopathologists will rarely classify a subject as solely “F4” - in practice a histopathologist is quite likely to give an opinion of “nodes of F4 within F3” or similar advice. For the purposes of this invention, F3/F4 patients are grouped together since the biomarker tests of the invention advantageously identify patients in this F3/F4 category. For the avoidance of doubt F3/F4 means advanced fibrosis.

The only treatments available for F3/F4 patients are liver transplantation.

F3/F4 subjects are also at enhanced risk of HCC and therefore are prescribed an intensive screening such as 6 monthly CP ultrasound screening.

Subjects with F3/F4 fibrosis are subject to enhanced surveillance. Thus if a patient is identified as having F3/F4 fibrosis, they may be prescribed a hospital consultation every 3-6 months. They may be prescribed aggressive lifestyle modification. This may include prescription of alternate diet. This may include prescription of blood sugar control measures.

Subjects with F3/F4 fibrosis are often at risk of cardiovascular morbidity which is enhanced in F3/F4 subjects. Therefore if a patient is determined to have F3/F4 fibrosis they may be prescribed hypertension management. Subjects diagnosed with F3/F4 fibrosis may be prescribed statins for hypercholesterolemia.

Patients with F3/F4 fibrosis may be at risk from diabetes. Patients identified as having F3/F4 fibrosis may be prescribed GLP analogues (for example a weekly injection of GLP analogue such as supplied by Novo Nordisk). Suitably if a patient is identified as having F3/F4 fibrosis then one or more GLP analogue(s) is administered to said patient. Most suitably if a patient is identified as having F3/F4 fibrosis and is diabetic then one or more GLP analogue(s) is administered to said patient.

In more detail, GLP analogues may be referred to as GLP-1′s or GLP-1 analogues (incretin mimetics). This type of medication works by increasing the levels of hormones called ‘incretins’. These hormones help the body produce more insulin only when needed and reduce the amount of glucose being produced by the liver when it’s not needed. They reduce the rate at which the stomach digests food and empties, and can also reduce appetite. There are six medications in the Incretin mimetic/GLP-1 analogues family:

| | |
|---|---|
| Generic or proper name | Brand or trade name |
| Exenatide (twice-daily injection) | Byetta |
| Exenatide (once-weekly injection) | Bydureon |
| Liraglutide (once-daily injection) | Victoza |
| Lixisenatide (once-daily injection) | Lixumia |
| Dulaglutide (once-weekly injection) | Trulicity |
| Semaglutide (once weekly injection) | Ozempic |

Thus patients identified as having F3/F4 fibrosis may be prescribed GLP analogue/incretin mimetic selected from these six medications. Suitably if a patient is identified as having F3/F4 fibrosis then one or more GLP analogue(s) selected from these six medications is administered to said patient. Most suitably if a patient is identified as having F3/F4 fibrosis and is diabetic then one or more GLP analogue(s) selected from these six medications is administered to said patient.

In tertiary care hospitals clinical trials are available for treating NAFLD which are using drugs for example FXR inhibitor (ObeLicholic Acid) in subjects with compensated cirrhosis due to nonalcoholic steatohepatitis (e.g. the REVERSE study by intercept). Thus patients identified as having F3/F4 fibrosis maybe prescribed FXR inhibitor (e.g. Obeticholic Acid). Suitably if a patient is identified as having F3/F4 fibrosis then FXR inhibitor (e.g. Obeticholic Acid) is administered to said patient.

Management can change from fewer visits to clinics to more visits to improve adherence to lifestyle changes. Thus patients identified as having F3/F4 fibrosis may be prescribed greater adherence to lifestyle changes, and/or may be prescribed more visits to clinics to improve adherence to lifestyle changes.

Suitably if a patient is identified as having F3/F4 fibrosis then said patient maybe prescribed or administered HCC surveillance (Hepato Cellular Carcinoma or liver cancer). Suitably if a patient is identified as having F3/F4 fibrosis then said patient may be prescribed or administered 6 monthly ultrasounds, and/or frequent blood tests and/or endoscopy.

In contrast, patients with F0-F2 fibrosis would be typically prescribed yearly surveillance. Thus they may be prescribed a hospital consultation every year or every 12 months. Patients with F0-F2 fibrosis may be prescribed alternative diet. Patients with F0-F2 fibrosis may be prescribed lifestyle changes such as exercise regimes and/or calorie control.

In one embodiment, if a subject is not determined to have advanced/severe fibrosis (i.e. not determined to have F3/F4 fibrosis) using the methods of the invention, then it is inferred that they have F0/F2 fibrosis.

### Fibrosis/Reference Sample

Suitably the methods of the invention are not carried out with reference to healthy controls or to subjects having no fibrosis (F0 fibrosis).

It is important to make comparisons to subjects having mild/moderate fibrosis because that enhances the performance of the method. The method is intended for use on patients suspected of having advanced or severe (F3/F4) fibrosis. The method of the invention is not likely to be economically valuable for mass screening of a healthy population. Suitably the methods of the invention are targeted to subjects suspected of having, or at risk of having, liver fibrosis.

Thus in one aspect a suitable subject having mild/moderate liver fibrosis to use as a reference value for the methods of the invention may be: 55 year old female with biopsy proven mild fibrosis, non hypertensive, non-diabetic with a BMI of 27.5.

Suitably cut off values from this subject may be used as reference values for comparison with the values from subject(s) being investigated using the methods of the invention as follows:

| | |
|---|---|
| Biomarker | Cut off Value 55 year old female |
| Glutamate | 34 µmol/L |
| CPS1 | 5 ng/ml |
| Arginine | 73 µmol/L |
| Citrulline/Ornithine Ratio | 0.45 |
| Reserve Capacity | 109 pmol/min |

More suitably in another aspect average values from a substantial cohort of F1-F2 subjects may be used as a reference value for the methods of the invention. This provides the advantage that any subject -to- subject variability is ‘averaged out’ by use of values derived from a substantial cohort of subjects. Thus more suitably cut off values maybe used for comparison with the values from subject(s) being investigated using the methods of the invention as follows:

| | |
|---|---|
| Biomarker | Cut off Value F1-F2 cohort |
| Glutamate | 64 µmol/L |
| CPS1 | 2.7 ng/ml |
| Arginine | 62 µmol/L |
| Citrulline/Ornithine Ratio | 0.41 |
| Reserve Capacity | 185 pmol/min |

Most suitably the methods in the invention are suitably carried out with reference to one or more matched subject(s) having mild to moderate fibrosis (F1 to F2 fibrosis). ‘Matched’ means matched to the subject of interest. Thus a suitable subject having mild/moderate liver fibrosis and matched to the subject of interest on as many as possible other clinical parameters is most suitably used for the comparison. In other words the biomarker/metabolite determinations (measurements) for the subject of interest are suitably compared to the biomarker/metabolite determinations (measurements) for a subject having mild to moderate fibrosis (F1 to F2 fibrosis) and matched on as many as possible clinical parameters such as hypertension, diabetes, or other characteristics.

Most suitably the reference/comparison subject having mild to moderate fibrosis (F1 to F2 fibrosis) may also be matched to the subject of interest on parameters such as age, sex, and/or BMI. However, it is a specific advantage of the invention that the method (i.e. the panels of biomarkers) are robust and are not confounded by parameters of age, sex or BMI. Thus, matching on age, sex and/or BMI is optional at the choice of the operator.

Thus most suitably cut off values from such a matched subject may be used as reference values for comparison with the values from subject(s) being investigated using the methods of the invention. These values may be determined in tandem/in parallel with the determination made for the subject of interest, or figures previously determined for the matched subject may be compared to the figures determined for the subject of interest.

### Further Advantages

To the best of the inventor’s knowledge and belief, nobody has undertaken bioenergetic analysis together with metabolite analysis. One reason is that there is a bias in the art against bioenergetic analysis. It is thought that this must be done quickly – within 1 to 2 hours for PBMC’s - it is thought that cells cannot be stored and so this is thought of as a poor technique in the art. It must be emphasised that it is not a natural logical step to combine these two fields. Typically scientists studying mitochondrial biology do not undertake metabolite analysis. The inventor assets that these are different fields and are not normally combined. This is a further indication towards the inventive step of this innovative method.

Moreover, it should be noted that there is no omittable drug for obesity/metabolic syndrome. Thus the person skilled in the art has no interest and no motivation towards combing bioenergetic and metabolite analysis from knowledge of the prior art. Added to this, the technical burden and challenge of carrying out untargeted metabolomics is very off putting as well as being technically demanding and expensive. Nobody skilled in the art is likely to undertake this purely on a speculative approach.

It should be emphasised that the inventor expended considerable intellectual effort in trying to draw or map or model of all of the different biochemical cycles which might have been underlying their observations. There is a clear inspirational moment when the inventors spotted the overlap between conventionally diverse areas of biology. In addition, the inventors brought a detailed understanding of the biochemistry such as the hydroxylation blockade which they believed they were observing. Thus, the inventors carefully chose what to test in their analysis, in contrast to a blind or speculative “shot gun” approach which might have been pursued without knowledge of the invention. Thus, even choosing what to test involved considerable intellectual and inventive effort.

In addition to this, the inventor brought substantial experience of diabetes and mitochondrial biology to the new field of liver disease and so has combined different and very distant fields of expertise in arriving at the invention.

Notwithstanding all of the above, it should be noted that nobody has done metabolomics for fibrosis before. Whatever disclosures have been made regarding the technique more generally, those are only involved in mouse studies and not humans, and have not involved any clinical studies, which further illustrates how the inventors have broken new ground in arriving at the invention.

Of course the compounds/biomarkers themselves are known. For example, CPS1 has been mentioned in proteomic studies in liver disease. However, it has never been used as a biomarker and no relationship to fibrosis has ever been disclosed or suggested. In addition, fibrosis is merely one example of a huge and diverse range of liver diseases so even if CPS1 has been observed in liver disease proteomics generally, there is still no teaching of its use in a specific panel of biomarkers and their specific diagnosis/inference of advanced or severe liver fibrosis.

Similarly, energetics have never been studied in NASH/NAFLD patients. Previously those techniques have only been used in unrelated disciplines such as diabetes, ageing or exercise tolerance. There has never been any connection of this type of study with liver disease before this invention.

Wong et al 2018 (ibid.) state on p 467: “Summary and recommendations CK18 is the most extensively evaluated test for NASH diagnosis, but overall accuracy is moderate at best. Although other biomarkers or panels might hold promise, most have not been independently validated. At present, none of the NASH biomarkers are ready for routine clinical use. However, active research in this field will further inform practice. The use of different NASH biomarkers in clinical trials depends on the mechanism of action of the study drugs. For example, cell death markers might be more relevant for agents targeting hepatocyte apoptosis⁵⁶. Biomarkers well suited for assessing metabolic changes, apoptosis or cell death, inflammation or fibrogenesis are therefore of greatest relevance.”

The mention of possible use of biomarkers for evaluation of metabolic changes (as for NAFLD progression but metabolic changes) is a very wide term and a vague statement with literally hundreds of pathways and related metabolites that might be affected. In the present invention, the panels of biomarkers disclosed have been evaluated by an innovative global metabolomic approach in which specific pathways and metabolites are not targeted. The highly significant metabolites are correlated with degree of biopsy proven fibrosis. The remark in Wong et al, is no more than a general hypothesis related to the possible association of NAFLD with metabolic changes and does not propose specific pathway(s) or metabolite(s). The comment in Wong et al serves as no more than an invitation to undertake a research project, as it provides no definite teaching. Also in arriving at the current invention, the inventor used two different approaches which were combined (using metabolomics and bioenergetics together) instead of just focusing on one field such as metabolic changes. Thus the inventive step/non-obviousness of the invention can be appreciated.

De Chiara et al 2018 (J.Hepatol. 2018 vol 69 pages 905-915) discloses that the CPS1 level changes in HFHC animals compared to controls (see FIG. 2 of De Chiara). However, this paper addresses dysregulation of the urea cycle in NAFLD and is focused on mechanistics of urea cycle dysregulation. De Chiara investigated gene and protein expression of CPS1 in HFHC animals and showed decreased activity and expression of CPS1. The inventor’s approach was different as the inventor measured plasma expression of CPS1 which was increased indicating mitochondrial matrix protein CPS1 is a marker for apoptotic and necrotic forms of hepatocyte death and injury and is released in the circulation after liver injury or damage. The inventor’s innovative research teaches that measurement of plasma CPS-1 can be used as a surrogate marker/biomarker for liver damage in NAFLD and hence a non-invasive biomarker for disease progression in NAFLD i.e. the teaching of the invention that CPS-1 (i.e. CPS-1 levels especially in blood, serum or plasma most suitably plasma) can be used as a biomarker for increased likelihood of having advanced or severe (F3/F4) fibrosis of the liver is an inventive/non-obvious advance over what was known.

### SAMPLE

Suitably the sample comprises biological fluid from the subject of interest.

Suitably the sample consists essentially of biological fluid from the subject of interest.

Suitably the sample consists of biological fluid from the subject of interest.

Suitably the sample comprises whole blood.

Blood has different fractions such as plasma, serum and cells. Suitably the sample comprises a blood fraction. Techniques for separation of different fractions such as plasma, serum, cells (e.g. PBMCs) from blood are routine and well known in the art.

In one embodiment suitably the sample comprises plasma or serum. These sample types are especially suitable for metabolomic analysis e.g. determining the level of metabolites such as glutamate, arginine, citrulline/ornithine, and are especially suitable for determining the level of CPS-1 expression. In one embodiment when the sample comprises a blood sample, the step of “determining the level of glutamate/arginine in said sample” or “determining the citrulline/ornithine ratio in said sample” or “determining the level of CPS-1 expression” comprises the step of separating the plasma or serum from said blood sample, and determining the glutamate and/or arginine and/or citrulline and/or ornithine level(s) and/or the level of CPS-1 expression in said plasma or serum.

In one embodiment suitably the sample comprises peripheral blood mononuclear cells (PBMCs). This sample type is especially suitable for determining reserve capacity. In one embodiment when the sample comprises a blood sample the step of “determining the reserve capacity in said sample” comprises the step of separating the PBMCs from said blood sample, and determining the reserve capacity of said PBMCs. Separation of PBMCs from blood is routine and well known in the art.

Suitably the sample comprises plasma or serum from the subject of interest.

Suitably the sample consists essentially of plasma or serum from the subject of interest.

Suitably the sample consists of plasma or serum from the subject of interest.

Suitably the sample comprises PBMCs from the subject of interest.

Suitably the sample consists essentially of PBMCs from the subject of interest.

Suitably the sample consists of PBMCs from the subject of interest.

Suitably the sample is a biopsy such as biological fluid provided from the subject.

Suitably the method may involve collection of the sample.

More suitably, the method does not involve direct collection of the sample but is performed on an in vitro sample provided from the subject of interest.

In one embodiment the sample is an in vitro sample previously collected and in this embodiment suitably the invention does not involve interaction with the subject’s body. Suitably the sample is an in vitro sample previously obtained from the subject.

Suitably the method of the invention is performed on sample(s) such as in vitro sample(s) obtained from, or provided from, subjects.

Suitably the method is an in vitro method.

When the sample is, or comprises, or is derived from, blood suitably said sample may further comprise additional components helpful in preserving or improving the sample e.g. anticoagulant(s) e.g. heparin.

Suitably the sample comprises protein from the subject. Protein preparation is well known in the art. This is useful in (e.g.) ELISA assays for example in detection of expression of CPS-1.

Suitably the sample is from a subject suspected of having, or having, liver fibrosis.

Suitably the sample is from a subject having F1 liver fibrosis.

Suitably the sample is from a subject, having F2 liver fibrosis.

Suitably the sample is from a subject having F3 liver fibrosis.

Suitably the sample is from a subject having F4 liver fibrosis.

Most suitably the sample is from a subject previously identified as having F1-F2 liver fibrosis.

Suitably the sample is from a subject suspected of having, or having, metabolic syndrome.

Suitably the sample is from a subject suspected of having, or having, diabetes, preferably type 2 diabetes.

Suitably the sample is from a subject suspected of having, or having, Non-Alcoholic Fatty Liver Disease (NAFLD).

Suitably the sample is from a subject suspected of having, or having, Non-Alcoholic Steatohepatitis (NASH).

Suitably the method is an in vitro method.

### FURTHER APPLICATIONS

The changes in the biomarkers in the panel (e.g. metabolite changes) can be utilised easily as a blood test such as a diagnostic blood test. Suitably this is combined with bioenergetic analysis (most suitably reserve capacity) using suitable instruments / techniques.

The methods described herein can be incorporated into a kit and/or a kit method for easy use. Thus in one embodiment the invention relates to a kit comprising reagent(s) for detection of the markers described in a method according to the present invention. Separate kits are described - one comprising reagent(s) for detection of the markers (such as the panel of markers) which are increased and one comprising reagent(s) for detection of the markers (such as the panel of markers) which are decreased. In addition a combination kit is disclosed, which combination kit suitably comprises reagent(s) for detection of both the markers (such as the panel of markers) which are increased and reagent(s) for detection of the markers (such as the panel of markers) which are decreased.

In one aspect the invention provides a kit comprising an agent which specifically detects the proteins or biomarkers of interest. The agents can be antibodies which can bind, suitably specifically bind, these biomarkers (most suitably one antibody per biomarker/one biomarker per antibody). The antibodies may be used as an immunoassay such as ELISA, protein dot blot etc. Other methods for measuring proteins can also be used such as spectrometry, absorbance, protein immunostaining, western blot etc. Amongst other things, the invention provides new use of known reagents as described herein.

Suitably the method and/or the kit of the invention are for use by clinicians e.g. in hospitals and/or doctors (physicians) e.g. in general practice (GP) setting such as a doctor’s surgery.

In a broad aspect the method comprises of using one or more biomarkers in a biological sample of patients with NAFLD. The markers can be used alone or in conjunction with other standard of care diagnostic aids to diagnose and stage the progression of NAFLD. This can lead to appropriate management plan.

This invention can be used as standard of care blood test in hospitals and also as a kit that can measure the biomarkers in the biological sample.

This invention can be used as a one-point quick test or kit. In this embodiment suitably detection/assay of markers is via Immunohistochemistry or ELISA.

For the panel with increased expression of metabolites (i.e. the panel including glutamate and CPS-1), metabolites can be measured as a quick test (semi quantitative test). The invention provides a kit comprising colourimetric test(s) for glutamate. Strips reactions are based on Glutamate dehydrogenase catalysed oxidation of glutamate in which the formed NADH reduces a chromogenic reagent. CPS-1 can also be measured by immunoassay.

For the panel with decreased expression of metabolites (i.e. the panel including arginine and reserve capacity), metabolites can be easily measured by kit or strips. The invention provides a kit comprising colourimetric test(s) for arginine. However it will be noted that reserve capacity is most preferably measured on Seahorse (see examples) or by fluorescent plates which has the advantage of being a quicker method. Such plates may be used with bloods samples depending on operator choice.

Further particular and preferred aspects are set out in the accompanying independent and dependent claims. Features of the dependent claims may be combined with features of the independent claims as appropriate, and in combinations other than those explicitly set out in the claims.

Where an apparatus feature is described as being operable to provide a function, it will be appreciated that this includes an apparatus feature which provides that function or which is adapted or configured to provide that function.

### BRIEF DESCRIPTION OF THE DRAWINGS

The present invention will now be described by way of example, with reference to the accompanying drawings, in which:

FIG. 1 shows graphs; the fundamental parameters of mitochondrial function measured by XF cell mito stress test kit and a representative of a run of the experiment. Cellular mitochondrial profile in human PBMCs of NAFLD patient with mild /moderate fibrosis (blue) and severe fibrosis (red). Well-defined inhibitors, oligomycin (Oligo), FCCP and Rot/antimycin A (AntiA) are used in mito stress kit. PBMCs were counted and seeded at density of 300,000 cells/well. Using Seahorse XFp analyser, three measurement of basal respiration were taken, followed by an injection of oligomycin (0.75 µM final) which indicated the ATP linked respiration. To assess maximal respiration, FCCP was injection at the 0.75 µM working concentration. Reserve capacity was measured as a difference between maximal and basal respiration. Representative data shown as a Mean ± SEM, n=3 replicates per sample.

FIG. 2 shows bar charts - Mitochondrial dysfunction in live peripheral blood mononuclear cells (PBMCs) from NAFLD patients with severe fibrosis. PBMCs from HC (N= 5), mild/moderate liver fibrosis (N= 10) and patients with severe fibrosis (N=10) were isolated, seeded at 3×10⁵ cells/well, and the Seahorse XFp extracellular flux analyzer was used to measure Basal respiration (A) Maximal respiration (B) ATP linked respiration (C) Reserve capacity (D) Non mitochondria production (E) and Proton leak (F). Data presented as mean ± SEM and analysed by one-way ANOVA with Tukey’s test where *p<0.05, **p < 0.01

FIG. 3 shows bar charts – Glycolysis as represented by ECAR

FIG. 4 shows bar charts - Bioenergetic Health Index (Bill)

FIG. 5 shows bar charts - Phenotype Stress test showing metabolic potential

FIG. 6 shows bar charts - Citrulline/Ornithine Reversal in advanced Fibrosis in NAFLD

FIG. 7 shows bar charts - Reduced Arginine in advanced Fibrosis in NAFLD

FIGS. 8 shows plots; FIG. 8a - Principle Component Analysis to Detect Outliers. Heat map with Hierarchical Clustering discovers KCH-050319-18 as an Outlier; FIG. 8b - Partial least squares discriminant analysis Scores plot comparing healthy control (HC) plasma v NAFLD. R₂ = 0.6, Q₂ = 0.45, AUROC 0.96, CV ANOVA p<0.01.

FIG. 9 shows a bar chart - CPS-1 Levels by ELISA

FIG. 10 shows bar charts - Circulating Markers of Inflammation and Oxidative Stress in healthy controls, NAFLD patients with mild/moderate fibrosis and severe fibrosis. Plasma concentrations of (A) Interleukin-6 (IL-6) (B) lnterleukin-8 (IL-8) (C) TNF-alpha (D) Interleukin-13 (IL-13). Data are represented as means ± SEM (HC n=9, F1-F2 n=12, F3-F4 =8), *p < 0.05. **p < 0.01 and ***p <0.001

FIGS. 11 shows graphs - FIG. 11a: ROC curve analysis using glutamate + CPS-1 in F1-F2 versus F3-F4 NAFLD groups showed sensitivity of 0.95 and p=0.0007; FIG. 11b: ROC curve analysis using Citrulline/Ornithine + Arginine + Reserve capacity in F1-F2 versus F3-F4 NAFLD groups showed sensitivity of 0.94 and p=0.0006

FIG. 12 shows graphs.

### EXAMPLES

### Example 1

### Methods

In this example, we investigated 30 subjects divided into 3 groups: subjects with NAFLD having mild fibrosis (n=10), subjects with NAFLD having severe fibrosis (n=10) and healthy controls (n=10). All NAFLD patients had biopsy proven fibrosis. Oxygen consumption rate (OCR) was measured in PBMCs using XFp Seahorse analyser. A mass spectrometric based untargeted metabolomic approach was used to analyze a broad range of human plasma metabolites in these samples. ELISA for CPS-1 and MSD for inflammatory markers was also done on corresponding plasma samples.

### Findings

The mitochondrial bioenergetics showed reduced basal respiration, ATP linked respiration, maximal respiration and reserve capacity in PBMCs of NAFLD patients with severe fibrosis compared to NAFLD patients with mild fibrosis. The untargeted global metabolomic approach showed 13 metabolites which were significantly different between the mild and severe fibrosis of NAFLD patients. Most of these metabolites were involved in the pathways occurring in mitochondria.

### Interpretation

Metabolites and bioenergetics measurements in peripheral blood samples can be incorporated together as a unique panel for diagnosing and staging of NAFLD.

### Study Design and Participants

Patients were recruited between 2018-2019 with written informed consent from Kings College Hospital clinics under ethical approval from the regional Research Ethics Committee (REC; ref number 18/LO/1355). The cross-sectional study adhered to the Ethical Principles for Medical Research Involving Human Subjects, World Medical Association Declaration of Helsinki. The patients were studied in 3 groups: Group 1. Healthy control (N=10) who had no history of any chronic disease were recruited with informed consent, and were age and sex matched with the patient study group. Group 2 included patient with steatosis with mild/moderate fibrosis stage 1 or 2 (N=10) and group 3 were patient with advanced fibrosis stage 3 or 4 (N=10). All subjects in group 2 and 3 had liver biopsy consistent with NASH (defined as the presence of at least grade 1 steatosis, hepatocellular ballooning, and lobular inflammation according to the NAFLD Activity Score [NAS]) and fibrosis (1-4) according to the NASH CRN classification). There was no prior history of decompensated liver disease, chronic HBV and HCV infection, HIV positivity, other causes of liver disease or history of a malignancy or any other serious co-morbidities.

The 20 participants with NAFLD were aged 20-74 (Mean=52) and the female: male ratio was 8:12. The average BMI (kg/m2) in the group was 35 ± 6.5. Out of the 20 subjects, 14 (70%) had type 2 diabetes and 10 (50%) were diagnosed with hypertension. The mean systolic blood pressure was 136 ± 14 (mmHg) and diastolic blood pressure was 81 ± 9(mmHg). Fibroscan value for liver stiffness measured as LSM value was 11± 5.2 kPa and the controlled attenuation parameter (CAP) applied to assess the fat content value was 331±74. Table 1 shows the baseline characteristics of subjects with NAFLD used in this study.

TABLE 1

Baseline characteristics of the NAFLD study cohort

| Variables | F1- F2 Fibrosis | F3-F4 Fibrosis |
|---|---|---|
| Age (years) | 49±15 | 54±14 (p=0.41) |
| Gender (female:male) | 3:7 | 5:5 |
| BMI (kg/m²) | 34±6 | 37±6(p=0.41) |
| HbA1c (%) | 6.8±1.2 | 6.8±1.3 (p=0.93) |
| HTN | 60% | 40% |
| DM | 70% | 70% |
| Systolic BP (mmHg) | 144±14 | 129±9* (p = 0.01) |
| Diastolic BP (mmHg) | 81±11 | 80±7 (p = 0.87) |
| Cholesterol (mmol/l) | 5.2±1.4 | 4.4±1.6 (p = 0.28) |
| Triglycerides | 2.2±0.9 | 2.6±1.6 (p = 0.49) |
| HDL | 1.1±0.3 | 1.1±0.3 (p = 0.67) |
| LDL | 3.1±1.2 | 1.8±1.0* (p = 0.03) |
| FBS | 139+62 | 125±41*(p = 0.60) |
| Bilirubin-total | 13±4 | 9+4* (p = 0.05) |
| ALP | 90±23 | 80±15 (p = 0.3) |
| ALT | 61±41 | 46±39 (p = 0.4) |
| AST | 37+1.4 | 32±19 (p = 0.55) |
| GGT | 80±89 | 66±37 (p = 0.7) |
| Albumin | 46±2 | 45±2 (p = 0.80) |
| INR | 1.1±0.1 | 1.0±0.1 (p = 0.21) |
| Platelets | 287±103 | 247±76 (p = 0.4) |
| Uric Acid | 399±59 | 329±54 (p = 0.08) |
| FIB-4 Score | 1.05±0.63 | 1.2±0.8 (p = 0.6) |
| Fibroscan LSM value (kPa) | 11.7+5.9 | 10.3±4.7 (p = 0.6) |
| Fibroscan CAP value (dB/m) | 357±45 | 310±89 (p = 0.15) |

Footnote: Data are means ± SD. BMI: Body Mass Index, HbA1c: Glycated haemoglobin, HTN: Hypertension, DM: Diabetes Mellitus, BP: Blood Pressure, HDL: High density lipoprotein, LDL: Low density lipoprotein, FBS: Fasting blood sugar, ALP: alkaline phosphatase, ALT: alanine aminotransferase, AST: aspartate amino-transferase, GGT: gamma glutamyl transferase. Key: *p<0.05

Currently no data is available to perform a formal sample size calculation for oxygen consumption rates in PBMCs of patients with NASH. The current study must be regarded as a pilot study. The aim of the study was to get an insight about the possible role of mitochondria dysfunction in the progression of simple steatosis to fibrosis in NAFLD. We estimated that 7-10 patients per group in NAFLD will be sufficient to get a reliable estimation of effect size. We proposed to include 5-10 healthy controls to establish reference values.

### Procedures

### Sample Preparation

Blood was collected in 8 ml Cell Preparation Tube (CPT) tubes (Becton Dickinson, Franklin Lakes, NJ, USA, ref. 362753) for separation of peripheral blood mononuclear cells (PBMCs) and plasma. The anticoagulant used in the tube is sodium heparin and cell separation is based on the principle using Ficoll method. The anticoagulated blood was collected by routine phlebotomy and tube inverted 8-10 times for mixing of anticoagulant and blood. The tubes were then centrifuged at room temperature (18-25° C.) in a horizontal rotor (swing-out head) for a minimum of 15 minutes at 1500 to 1800 x g. After centrifugation, PBMCs were visible as a whitish layer just under the plasma layer. The plasma was removed without disturbing the cell layer and separation of cells was done immediately following centrifugation for best results. After separation of PBMCs from plasma they were immediately counted, and viability checked with Countess Automated Cell Counter. PBMCs were plated on XFp 8-well of polystyrene plates designed for the Seahorse XFp analyser (Agilent Technologies Santa Clara, CA United States) within 2 hours. The plasma samples were immediately stored at -80° C. for metabolomic assays.

### Measurement of Bioenergetics in Peripheral Blood Mononuclear Cells (PBMCs)

Cellular bioenergetics was performed using XF cell mito stress test kit in a Seahorse XFp analyzer (Agilent Technologies). PBMCs were suspended in XF media and 300,000 cells/well were seeded to Cell-Tak (Beckton Dickinson Ltd) coated XFp plates (Agilent Technologies). All experiments were performed with 3 replicate wells in the Seahorse XFp analyzer.

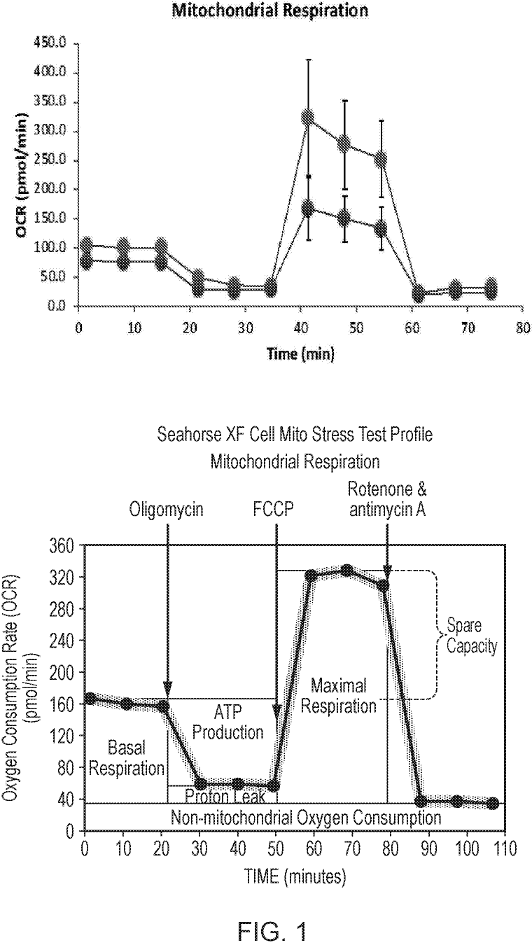

OCR, a measurement of mitochondrial respiration, and ECAR which correlates to number of protons released from the cell with potential contribution from glycolysis and the Krebs cycle, were measured in the presence of specific mitochondrial activators and inhibitors. Oligomycin (ATP synthase blocker) was used to measure ATP turnover and to determine proton leak, the mitochondrial un-coupler FCCP (carbonyl cyanide 4-[trifluoromethoxy] phenylhydrazone) was used to measure maximum respiratory function (maximal OCR). Reserve capacity was calculated as maximal OCR minus the basal respiration. At the end of the experiments, Rotenone (inhibitor of complex I) and Antimycin A (a blocker of complex III), were injected to completely shut the mitochondrial respiration down, to confirm that any changes observed in respiration were mitochondrial (Brand and Nicholls, 2011; Dranka et al., 2011) (FIG. 1)

For a measurement of basal respiration 3 measurements were taken before injecting ATP synthase inhibitor, Oligomycin at 0.75 µM (final concentration). FCCP was then injected at 0.75 µM. Finally, a mixture of rotenone and antimycin A (1 µM) was injected. Mitochondrial basal respiration, proton leak, spare capacity and maximal respiration were measured after correcting for non-mitochondrial respiration. OCR and ECAR rates were normalised to cell count for PBMCs. Cell Energy Phenotype Test Kit which measures metabolic potential of cells was also used in a subset of experiments. The XF Mito stress test report generator automatically calculate the XF cell mito stress test parameters from Wave data that was exported to Excel.

### Global Metabolomic Profiling:

A mass spectrometric based metabolomic approach based on the MxP® Global Profiling QUANT platform (Biocrates Life Sciences, Innsbruck, Austria) was used to analyze a broad range of human plasma metabolites covering various biochemical classes. A statistical method was applied to identify differences between the individual groups. The analyses included data normalization and transformation followed by univariate statistics with significance testing.

Plasma samples were extracted by a proprietary method and separated into lipid and polar fractions after precipitation of proteins (supplementary material; FIG. S1). Two types of mass spectrometry analysis were used for MxP® Global Profiling: Gas chromatography-mass spectrometry (GC-MS; Agilent 6890 GC coupled to an Agilent 5973 MS System, Agilent, Waldbronn, Germany) and Liquid chromatography-MS/MS (LCMS/MS; Agilent 1100 HPLC-System, Agilent, Waldbronn, Germany, coupled to an Applied Biosystems API4000 MS/MS-System, Applied Biosystems, Darmstadt, Germany) (van Ravenzwaay et al., 2007). For GC-MS analysis samples were sequentially derivatized before measurement. In LC-MS/MS analysis a Metanomics Health proprietary technology was applied which allows targeted and high sensitivity MRM (Multiple Reaction Monitoring) profiling in parallel to full screen analyses.

### Normalization

Pooled reference samples derived from aliquots of all plasma samples were run in parallel throughout the entire analytical process and used to assess analytical process variability as part of the rigorous quality control performed at the metabolite, sample and experiment (study) level. Subsequently, data were normalized against the median in the pool reference samples to give pool-normalized ratios (performed for each sample per metabolite). This compensated for inter- and intra-instrumental variation.

To enable the MxP® Boost quantification and to allow for an experiment-to-experiment alignment of data, the MxPool™ concept was devised. The MxPool™ is a defined stock of the sample material to be analyzed and is stored in-house at Metanomics Health. Aliquots of the MxPool™ are analyzed within each experimental batch and within each project and all data from that batch are normalized to the MxPool™ in addition to the pool material generated for said batch, i.e. an additional normalization step of already experiment pool-normalized metabolite ratios is performed.

### MxP® Boost Quantification

The metabolome of a large human plasma pool (MxPool™) was quantified by various methods, and results were cross-validated where possible. Up to now, more than 2.000 metabolites with absolute concentrations have been quantified in this material. Multiple aliquots of the MxPool TM material were measured within this project and used as one-point calibrator to yield metabolite concentrations. Metabolites that could not be quantified by this method were analyzed semi-quantitatively. Quality control is samples is given in supplementary section S1.

### Carbamoyl Phosphate Synthase 1, Mitochondrial (CPS1) in Plasma by ELISA

Plasma CPS-1 concentration were measured using a commercial sandwich enzyme-linked immunosorbent assay (Human Carbamoyl Phosphate Synthase 1, Mitochondrial (CPS1) ELISA Kit Catalog No: RD-CPS1-Hu, RedDot Biotech limited, Kelowna, Canada) as per the manufacturer’s instructions (supplementary section S2).

### Measuring Expression of Proinflammatory Cytokines

V-PLEX Proinflammatory Panel 1 Human Kit (Meso Scale Diagnostics, Rockville, USA) was used to investigate a range of cytokines namely IFN-γ, IL-1β, IL-2, IL-4, IL-6, IL-8, IL-10, IL-12p70), IL-13, TNF-α in patients with NASH and HC as per manufacturer’s instructions (supplementary section S3).

### Statistical Analysis

Statistical analysis was performed using GraphPad (GraphPad Software, Inc). The distribution of the data was tested using the Kolmogorov-Smirnov test (Graph pad) For parametric analysis, groups were compared using t-test (2 groups) or one-way ANOVA with post-hoc Tukey’s multiple comparison test (>2 groups). For non-parametric analysis, groups were compared using Mann-Whitney (2 groups) or Kruskal Wallis with Dunn’s post hoc test with Bonferroni correction (>2groups). Data was presented as mean ± Standard error of the mean.

Metabolomic data was analysed using univariate based on t-test and multi variate analysis comprising Principle Component Analysis (PCA) and Partial Least Squares-Discriminant Analysis (PLS-DA). Univariate statistics for the entire metabolomic dataset and each individual group to be analyzed statistically, the minimum, maximum, mean and median values were determined. Mean and median values were calculated on a logarithmic scale and then back-transformed to non-logarithmic scale. Variabilities within sample groups were evaluated by calculating the standard deviation of log10-transformed data. Subsequently, the standard deviation was transformed into a relative standard deviation (RSD) which was asymmetric due to the back-transformation from the logarithmic scale (i.e. RSD up and RSD down differ). The RSD down was calculated according to: RSD down =1-10^(-SDlog). The univariate analysis comprised one-way Analysis of Variance (ANOVA) and Tukey Honest Significant Difference (HSD) as post-hoc analysis. In contrast to multivariate analyses, single metabolite univariate models consider each metabolite independently. Accordingly, the results are not influenced by how many and which other metabolites are measured. In a first step, one-way ANOVA was used to assess whether the estimated sample means between the experimental groups differ from each other. This analysis revealed that the potential confounders gender and age had a significant effect on the data. Therefore, they were included in the univariate statistical model. Accordingly, the ANOVA model used GROUP, GENDER and AGE as factors, with GROUP being the factor of interest. In order to determine which of the multiple group comparisons produce significant differences, Tukey’s HSD test was applied as post-hoc analysis. The Tukey HSD test simultaneously compares all possible pairs of group means and additionally corrects for the type I error rate (Tukey, 1949).

### Results

### Mitochondrial Dysfunction in Patients With Advanced Fibrosis in NASH

We assessed bioenergetics in 3 groups: Healthy controls (N=5), patients of NAFLD with mild to moderate fibrosis (F1-F2) and patients of NAFLD with severe fibrosis (F3-F4).

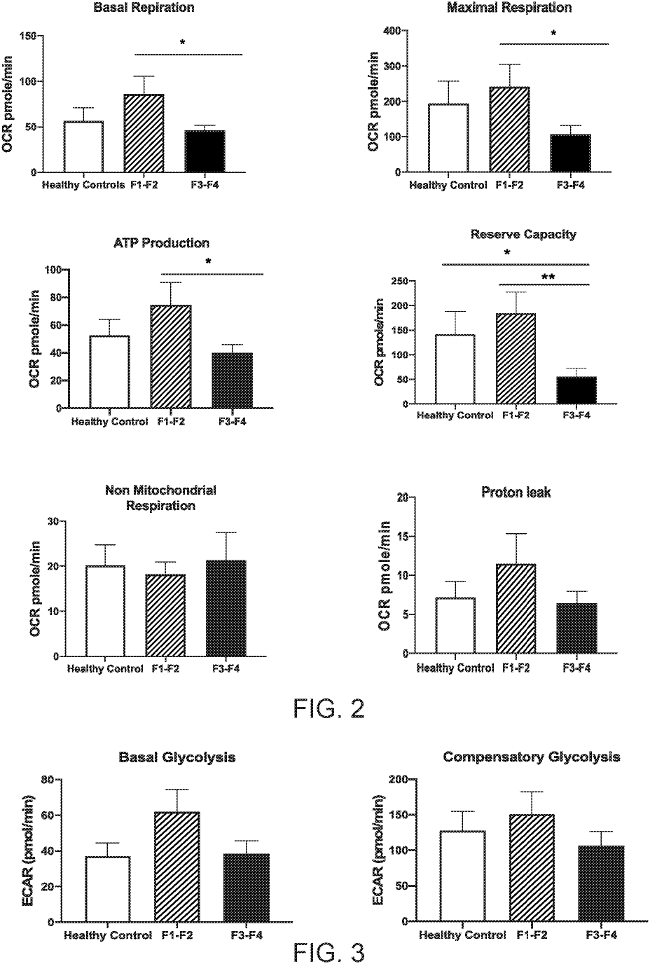

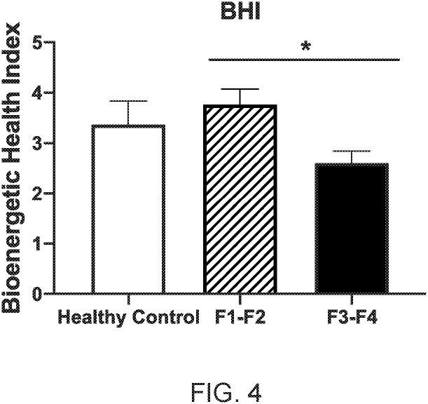

The basal respiration (FIG. 2.1), ATP linked respiration (FIG. 2.2), maximal respiration (FIG. 2.3) and reserve capacity (FIG. 2.4) were all reduced in PBMCs of patients with severe fibrosis. Proton leak, non-mitochondrial respiration (FIG. 2.5 and FIG. 2.6) and ECAR (FIG. 3) were similar in the 3 groups. These data suggest that in patients with severe liver fibrosis there is mitochondrial dysfunction as manifested by a significant suppression of all mitochondrial parameters.

Basal respiration is the energetic demand of the cell under baseline conditions and reduction in this indicate that oxygen consumption used to meet cellular ATP demand is compromised in severe fibrosis (45 ± 6, n= 9) as compared to patients having mild fibrosis (86 ± 19, n= 7, p = 0.04, FIG. 2.1). Our results show that ATP-linked respiration by the mitochondria that contributes to meeting the energetic needs of the cell is also significantly reduced in severe fibrosis (40 ± 5, n= 9) versus mild/moderate fibrosis (74 ± 16, n= 7, p = 0.04) (FIG. 2). There was significant reduction in maximal respiration between mild/moderate fibrosis (242 ± 62, n= 7) and patients with F3 and F4 fibrosis (106 ± 25, n= 9, p ≥ 0.05) (FIG. 2). The reserve capacity was also significantly reduced in severe fibrosis (56 ± 16, n= 9) as compared to mild/moderate fibrosis (184 ± 42, n= 7, p = 0.0064) (FIG. 2).

The reduced reserve capacity and reduced maximal respiration are suggestive of a compromised response to stress in patients with advanced fibrosis.

### Reduced Bioenergetic Health Index in PBMCs of NASH Patients With Advanced Fibrosis

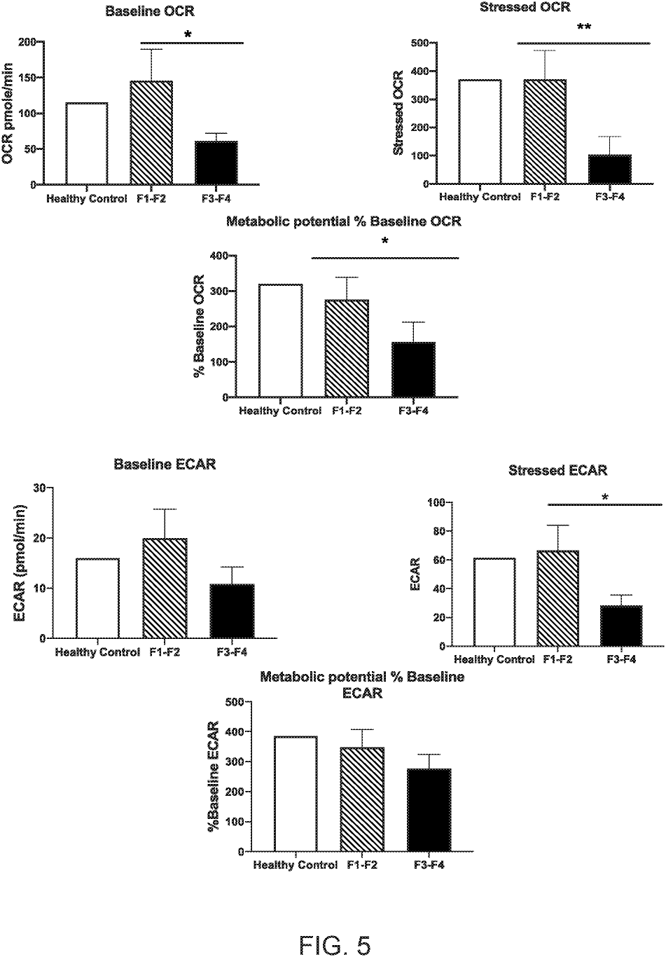

The Bioenergetic Health Index (BHI) is a dynamic measure of the response of the body to stress. It is indicative of the dysfunctional metabolic response and any defects in the electron transport chain (ETC) will result in a lower BHI because of lower reserve capacity, ATP-linked respiration or increased uncoupling. BHI was calculated by using the bioenergetics data using the formula below as described by the Darley-Usmar group (Chacko et al., 2014): BHI= log (ATP-linked x reserve capacity)/ (proton leak x non-mitochondrial) The mean BHI value for patients with severe fibrosis (2.6 ± 0.2, n = 9, p = 0.0092) was significantly lower than in patients with mild/moderate fibrosis (3.7 ± 0.3, n == 6) (FIG. 4).

### Defects in Metabolic Switching

In a sub-set experiment (n=9) we used Cell Energy Phenotype Test Kit (Agilent Technologies) which measures mitochondrial respiration and glycolysis under baseline and stressed conditions, to reveal the three key parameters of cell energy metabolism: Baseline Phenotype, Stressed Phenotype, and Metabolic Potential. By simultaneously measuring the two major energy producing pathways in live cells - mitochondrial respiration and glycolysis, we can determine the energy phenotypes of cells in patients with mild/moderate and advanced fibrosis in NASH.

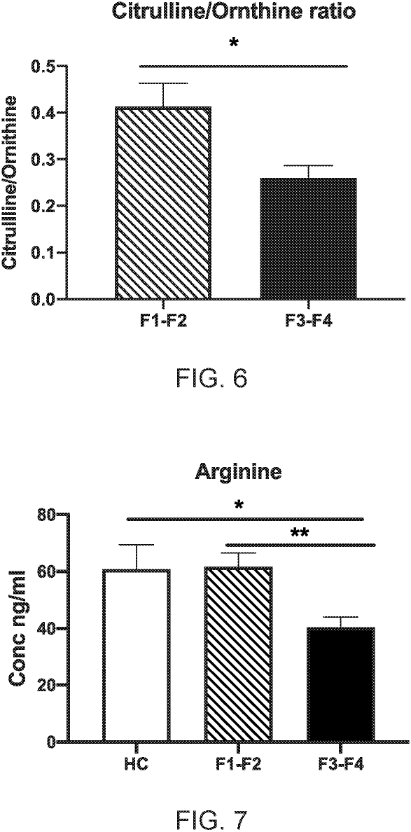

Our results show that in patients with F3/F4 fibrosis there is reduced basal OCR, reduced stressed OCR and ECAR and reduced metabolic potential for mitochondrial respiration (FIG. 5). The basal ECAR and the metabolic potential for glycolysis is not significantly different between NAFLD patients with the mild and severe fibrosis.

### Global Metabolomic Profiling Showing Altered Amino Acid Involved in Urea Cycle

Global untargeted metabolomics was performed on HC (n=9), patients with NAFLD with F1-F2 fibrosis (n=10) and F3-F4 fibrosis (n=10) using MxP® Global Profiling. Metabolite concentrations of each sample were determined in a single analysis. In the present study, we acquired data for a total of 493 metabolites, of which 401 were known metabolites and 92 unknown analytes. Furthermore, 25 metabolite to-metabolite ratios and sums were calculated and included in the statistical analyses. Univariate analysis was performed between the three groups.

Comparing mild/moderate with severe liver fibrosis in patients with NAFLD, 13 metabolites were significantly changed (p-value (F-Stats) < 0.05 and p-value (Tukey) < 0.05) out of the wide range of untargeted metabolomics data, (table 2). Out of these 13 metabolites, 5 are amino acids, 3 are choline ether lipids, 2 complex fatty acid lipids, 1 carbohydrate and 3 unknown metabolites (Table 2).

TABLE 2

Metabolites significantly altered while comparing mild/moderate fibrosis NAFLD patients with severe fibrosis

| No | Metabolites (p-value (F-Stats) < 0.05 and p-value (Tukey) < 0.05) |
|---|---|
| 1 | Glutamate |
| 2 | Unknown polar |
| 3 | Choline ether lipid (C40:5) |
| 4 | Choline ether lipid (C42:4) |
| 5 | Maltose |
| 6 | Unknown lipid |
| 7 | Citrulline / Ornithine |
| 8 | Histidine |
| 9 | Choline ether lipid (C44:6) |
| 10 | Sphingomyelin (C41:3) |
| 11 | Unknown lipid |
| 12 | Sum of Branched Amino Acids / Sum of Aromatic Amino Acids |
| 13 | Arginine |
| 14 | Phosphatidylcholine (C40:1) |

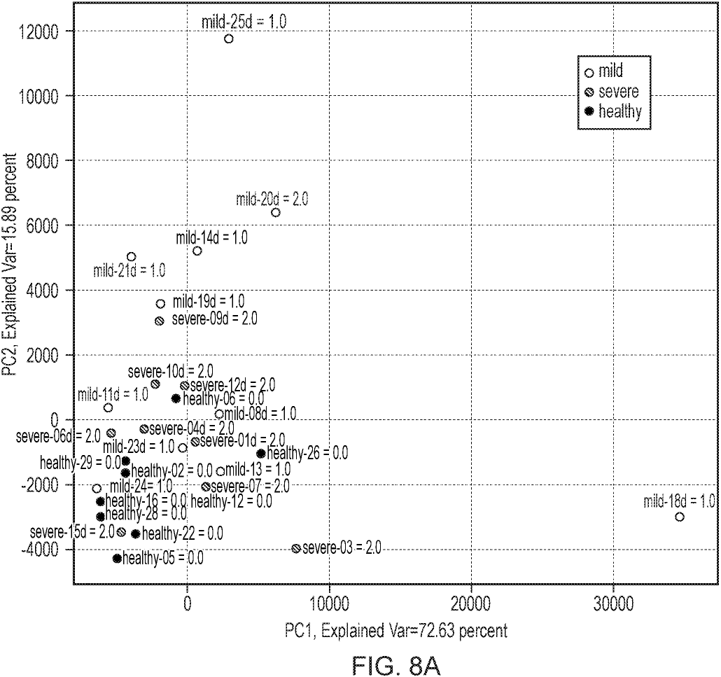

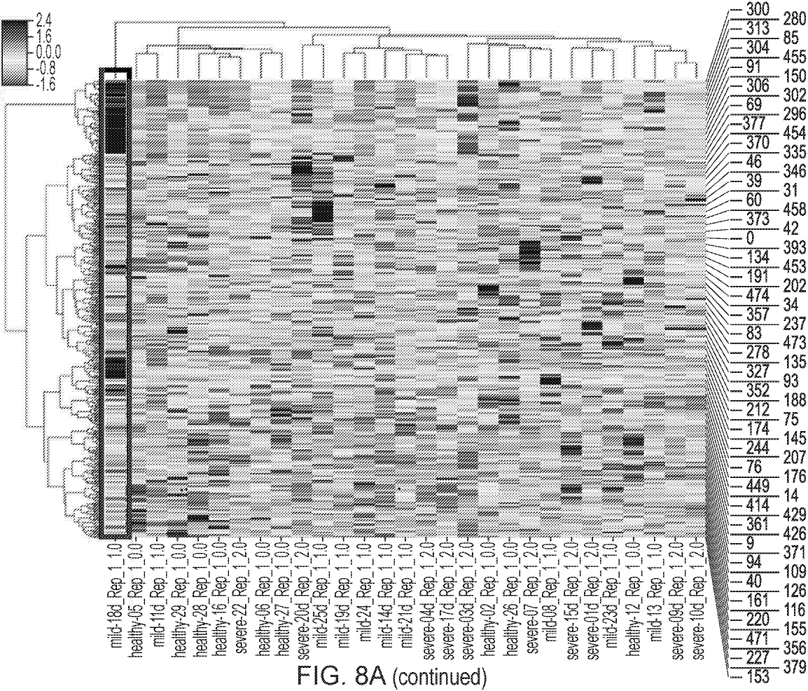

Interestingly all the 5 amino acids which were significantly increased or decreased between mild/moderate fibrosis and severe fibrosis were part of urea cycle related to mitochondria. The citrulline/ ornithine ratio was reduced in severe fibrosis (0.26 ± 0.08, n = 10) compared to mild fibrosis (0.41 ± 0.15, n = 10, p = 0.02). Citrulline was low in severe fibrosis and ornithine was high. This was reversed in mild/moderate fibrosis with low ornithine and high citrulline (FIG. 6). Citrulline and ornithine are alpha amino acids and are part of Urea cycle in the liver. The conversion of ornithine to citrulline is the rate limiting step of urea cycle and occurs exclusively in mitochondria as compare to rest of the steps occurring in cytosol. Mitochondrial dysfunction can result in the disruption of the urea cycle and hence changes in the metabolites involved such as ornithine and citrulline. Arginine another component of urea cycle is also significantly reduced in severe fibrosis (40.4 ± 11.32, n = 10, p = 0.002) as compared to mild/moderate fibrosis (61.68 ± 15.22, n = 10) (FIG. 7). Histidine and Glutamate were upregulated in severe fibrosis (p < 0.001). Glutamate is a key compound in cellular metabolism and involved in both urea cycle and TCA cycle. Complex ether lipids which have antioxidant properties are also reduced in severe fibrosis. Maltose, a disachhride and precursor of glucose is high in F3-F4 fibrosis (p=0.01).

### Changes in Metabolites in HC and NASH Patients

Metabolites were also analyzed between HC and patients having NASH. Our collaborator REVIVEMED performed Hierarchical Clustering to remove any outlier. Only 1 sample was an outlier (Supplementary material). Using their platform, molecular networks associated with significantly dysregulated metabolites between Healthy and NASH patients are shown in table.

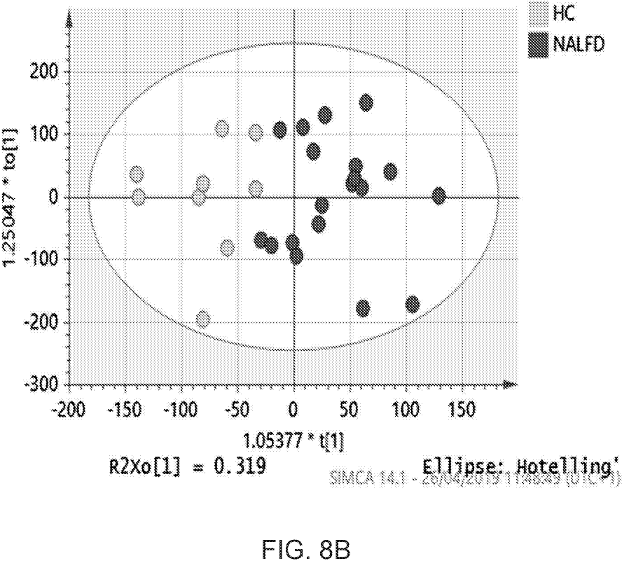

Using Partial Least Squares-Discriminant Analysis (PLS-DA), healthy controls and patients with NAFLD could be distinguished (FIGS. 8)

### 3.6: Increased Carbamoyl Phosphate synthetase-1(CPS-₁) Levels in Plasma Of Patients with Advanced Fibrosis in NASH

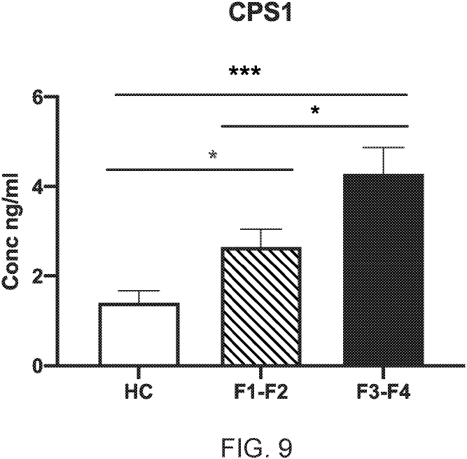

To further evaluate the changes in the metabolites of urea cycle especially the first-rate limiting step occurring in mitochondrial matrix we investigated the expression of CPS-1 levels in plasma of the corresponding patients. CPS-1 occurs in mitochondria and results in formation of carbamyl phosphatase which is utilized in conversion of ornithine to citrulline. As our results from metabolomics showed decreased citrulline and high ornithine in patients with high fibrosis we measured the plasma levels of CPS-1 to analyse any changes in this key enzyme of urea cycle. Plasma CPS₁ levels were measured in patients with NAFLD having different degrees of fibrosis and healthy controls using the ELISA kit for quantitative sandwich immunoassay technique. In comparison to the HC (n=9), the NASH patients with mild/moderate (n=10) and severe fibrosis (n=10) had significantly high CPS-₁ levels in plasma (HC versus F1-F2: p =0.02 and HC versus F₃-F₄: p= 0.0003) (FIG. 9). The CPS-₁ values were also significantly high in F₃-F₄ versus F₁-F₂ (p =0.02).

### 3.7: Increased Expression of Pro Inflammatory Cytokines in Severe Fibrosis In Patient with NASH

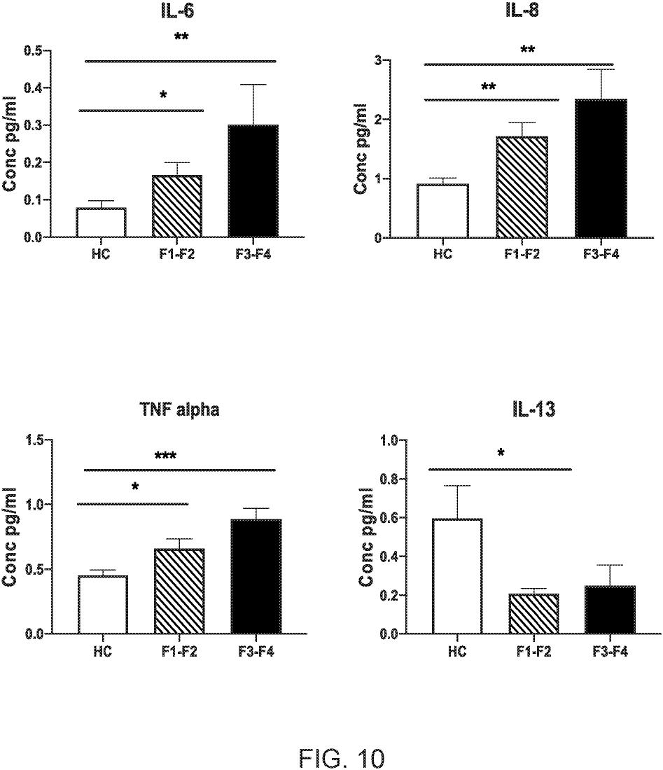

Proinflammatory human cytokines kit (Meso Scale Diagnostics, Rockville, USA) was used to investigate a range of cytokines namely IFN-γ, IL-1β, IL-2, IL-4, IL-6, IL-8, IL-10, IL-12p70, IL-13 and TNF-α to understand the systemic inflammatory response due to oxidative stress. Interestingly proinflammatory cytokines IL-6, IL-8 and TNF-α were significantly increased in NASH patients with fibrosis compared to HC (FIG. 10). IL-13 which is considered as anti-inflammatory cytokine was significantly higher in HC as compared to NASH patients. These results showed that the systemic response resulting from mitochondrial dysfunction cause a cascade of inflammatory reactions which also is evident in the cytokines.

### 3.8: Biomarker Panel for Liver Fibrosis in NASH Patients

Combining the bioenergetic and metabolomic data we analyzed two separate panel of biomarkers: panel with increase expression of metabolites and panel with decrease expression of metabolites.

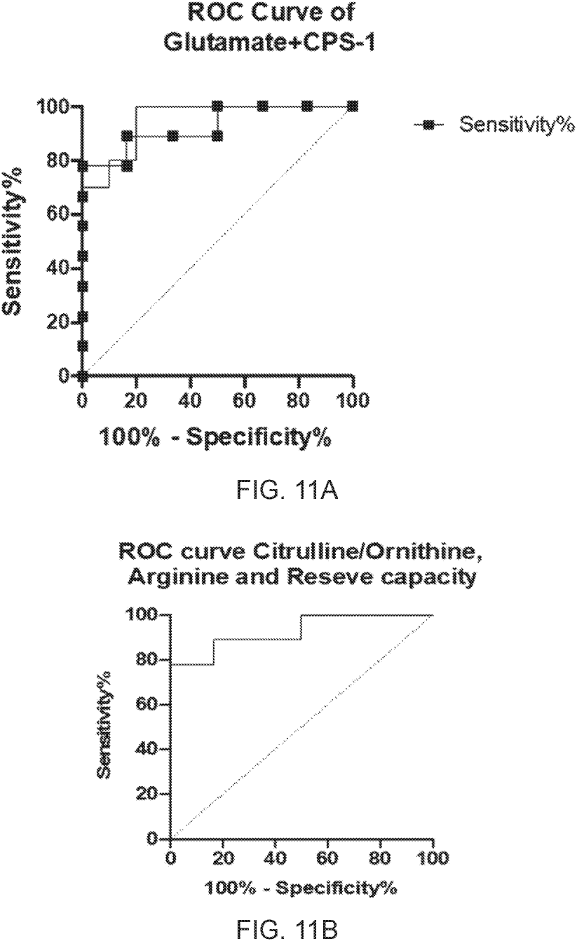

The panel for increased expression of metabolite included measuring glutamate and plasma levels of CPS-₁. The ROC curve analysis showed the sensitivity of 95% and p=0.0007 (F1-F2 versus F₃-F₄) FIG. 11-a.

The other panel with decreased metabolites was based on levels of citrulline/ornithine ratio, Arginine and bioenergetic value of reserve capacity. The ROC curve analysis showed the sensitivity of showed sensitivity of 0.94 and p=0.0006 (F₁-F₂ versus F₃-F₄) FIG. 11-b.

### Discussion

The main function of mitochondria is the generation of by oxidative phosphorylation, but apart from energy production mitochondria also have many other functions in cellular metabolism (Brand and Nicholas, 2011). As mitochondria regulate metabolic and energy homeostasis, its dysfunction is implicated in the pathophysiology of numerous chronic conditions such as obesity, insulin resistance, Type 2 Diabetes Mellitus (T2DM), diabetes-related complications (Holmstrom et al and Cjaka et al) and non-alcoholic fatty liver disease (NAFLD) (Perez-Carreras et al) etc. Maintaining normal mitochondrial function is critical for survival and maintenance of stable biological functions and cellular repair. We propose that mitochondrial dysfunction in hepatocytes can result in a systemic inflammatory response due to liver injury and oxidative damage. In the liver, this is accompanied with disturbance in the metabolic pathways which can be evaluated by measuring level of different metabolites in the peripheral blood. Mitochondrial functional changes cause impairment of fatty acid b-oxidation resulting in a vicious cycle of increased lipid intermediates, insulin resistance and reactive oxygen species leading to more inflammation and hepatocyte necrosis. These changes can be measured in peripheral cells due to a global immune response related to mitochondrial dysfunction in hepatocytes. Peripheral blood cells such as leucocytes and platelets can act as surrogate markers for different chronic diseases (A Perl et al, Cjaka et al, Rudkowska, I et al, Zharikov and Shiva).

In this study, we have shown for the first time an integrated approach for investigating mitochondrial function and untargeted metabolomics in patients with different stages of fibrosis in NAFLD. Our aim was to establish novel platform for non-invasive biomarkers in progression of NAFLD by using bioenergetics and metabolite changes.

The role of mitochondrial dysfunction in progression liver disease is not a new concept (Christie and Judah, 1954). Mitochondrial dysfunction in NASH has been hinted at histologically using electron microscopy where swollen and rounded hepatocellular mitochondria (Sanyal et al) were observed. However, with techniques to measure bioenergetics in live PBMCs it is possible to more fully study mitochondrial function. In our study, all parameters of mitochondrial function namely basal respiration, ATP linked respiration, maximal respiration and reserve capacity were significantly reduced in patients with severe (but not mild) fibrosis.

Reserve capacity serves to meet increase energy demands especially during periods of oxidative stress. Defects in the ETC can result in a lower BHI because of the lower reserve capacity, ATP-linked respiration or increased uncoupling. It is an indicator of bioenergetic health in real-time and can serve as prognostic value for identifying progressive deterioration in mitochondrial function. Metabolic potential for mitochondrial respiration was also reduced in advanced fibrosis but interestingly the potential for glycolysis was not significantly different between mild/moderate fibrosis and advanced fibrosis.

Mitochondria is an important cellular organelle as most significant metabolic pathways such as TCA cycle, beta-oxidation of fatty acids, rate-limiting steps of urea cycle, heme biosynthesis, cardiolipin synthesis, quinone and steroid biosynthesis occur completely or partly in the mitochondria. Progression of steatohepatitis likely involves both direct injury from excess fatty acids oxidation and increased oxidative stress within hepatocytes which forms the concept of double hits or insults to the hepatocyte. This creates a vicious cycle where fat accumulation causes defects in ETC leading to failure to generate reduced NAD and FAD, which further effects fatty acid oxidation and other metabolic pathways (Tarek Hussein).

Our untargeted metabolomic approach showed about 14 metabolites highly significant between the mild/moderate and severe fibrosis in NASH patients (Table 2). Out of these, five significant metabolites were amino acids which are all linked with the urea cycle or TCA. The citrulline/ornithine ratio was significantly reduced (P ≥ 0.05) in NASH patients with severe fibrosis (FIG. 7). Citrulline and ornithine are the most important metabolites in the Urea cycle pathway which is exclusively located in liver. This is the only rate limiting step in urea cycle which occurs inside mitochondria whereas rest of the steps of urea cycle are cytosolic. Recently De Chiara, Francesco et al. has shown that urea cycle enzymes are affected in rats models of NASH and humans resulting in hyperammonemia and impairment of urea synthesis. They suggested strategy of targeting ammonia as a potential treatment for NASH. In 2019, Canbay and Sowa also published a study suggesting the role ofl-ornithine 1-aspartate (LOLA) a known effective ammonia-lowering agent for treatment of NAFLD due to its actions of enhanced ammonia removal, increased anti-oxidative capacity, and attenuated lipid peroxidation by glutamine and glutathione and improved hepatic microcirculation due to 1-arginine-derived NO (Canbay and Sowa). K.L. Thomsen et al. also suggested hyperammonemia in NASH results in progression of fibrosis and hypothesized that treating ammonia can be a potential target for prevention of fibrosis progression of patients with NASH (K.L Thomsen et al).

The other metabolite significantly reduced in NASH patients with severe fibrosis was Arginine which also forms part of urea cycle. Arginine is hydrolyzed to form urea and ornithine. Reduced formation of Arginine can result in less urea production and more ammonia in the body. Thus, defects in urea cycle also effects the TCA which is a central driver of cellular respiration and vice versa. Glutamate another amino acid was significantly higher in NASH patients with severe fibrosis. Glutamate dehydrogenase (GDB) is an enzyme, present again only in the mitochondria and required for urea synthesis, that converts glutamate to α-ketoglutarate, and vice versa.

To further explore the role of metabolites involved in urea cycle as potential biomarkers for progression of fibrosis in NASH we measured CPS-₁ levels in plasma of the same set of NASH patients and HC by ELISA. CPS1 is the most abundant protein in liver mitochondria. Our results showed highly significant levels of CPS-₁ in plasma of NASH patients with severe fibrosis as compared to mild/moderate fibrosis and HC. CPS-₁ was also significantly higher in NASH patients with mild/moderate fibrosis as compared with the HC. Previously CPS1 has been found in serum or plasma of sepsis animal models and plasma of human septic patients, suggesting that it might serve as a serum marker for detecting mitochondrial injury of the liver under septic conditions (Crouser et al; Struck et al). Another study has also shown that the hepatocyte-selective and most abundant mitochondrial matrix protein CPS1 is a marker for apoptotic and necrotic forms of hepatocyte death and injury and is released in the circulation after acute liver injury (Sujith et al). El-Sheikh et al. has recently shown that the tissue and serum CPS-₁ correlated significantly in moderate and severe fibrosis in HCV patients and in these patients, there was significantly higher levels of serum CPS1 and lower mitochondrial counts than those with moderate fibrosis.

Proinflammatory cytokines IL-6, 1L-8 and TNF-a were significantly increased in NASH patients with fibrosis compared to HC indicating that mitochondrial dysfunction is accompanied with systemic immune response and utilizing combined approach of measuring mitochondrial bioenergetics and metabolomics we can elaborate novel biomarkers for progression of fibrosis in NASH. We propose platform of non-invasive biomarkers based on bioenergetics, metabolites involved in urea cycle notably citrulline/ornithine ratio, arginine, glutamate and CPS-₁ levels in plasma for identifying degree of fibrosis in NASH patients.

Confirmatory measurement of bioenergetics in corresponding liver tissue is taught to confirm that the changes in hepatocytes are accompanied by systemic changes in immune cells which can be utilized as non-invasive biomarkers for liver fibrosis.

In conclusion, this is the first study to show mitochondrial functional changes in peripheral cells with accompanied with changes in urea cycle metabolites which can clearly differentiate mild and severe fibrosis in NAFLD patients.

### Example 2 - Demonstrations of Sensitivity and Specificity

Combining the bioenergetic and metabolomic data we analysed two separate panels of biomarkers: panel with increased expression of metabolites and panel with decreased expression of metabolites.

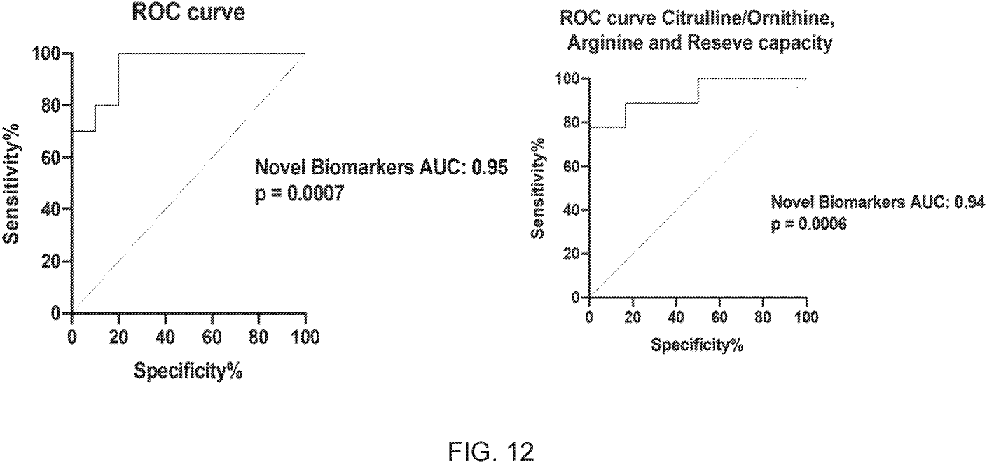

In this example the panel for increased metabolites included measuring glutamate and plasma levels of CPS-₁. The ROC curve analysis showed the sensitivity of 95% and p=0.0007 (F1-F2 versus F3-F4) See FIG. 12 (left graph).

In this example the panel for decreased metabolites was based on levels of citrulline/ornithine ratio, Arginine and bioenergetic value of reserve capacity. The ROC curve analysis showed the sensitivity of showed sensitivity of 0.94 and p=0.0006 (F1-F2 versus F3-F4) FIG. 12 (right graph).

In more detail in the graphs of FIG. 12 there is only one solid line on each and that shows Area Under the Curve (AUC) which shows relationship between sensitivity and specificity. So from FIG. 12 it can be seen that the ROC curves quantify the overall ability of the methods of the invention to discriminate between those individuals with the disease (advanced/severe fibrosis i.e. F3/F4) and those without the disease (mild or moderate i.e. F1/F2). The closer the curve follows the left-hand border and then the top border of the ROC space, the more accurate the test.

FIG. 12 (left) and 12 (right): ROC curve analysis using glutamate + CPS-₁ in F1-F2 versus F3-F4 NAFLD groups showed sensitivity of 0.95 and p=0.0007 and for Citrulline/Ornithine + Arginine + Reserve capacity in F1-F2 versus F3-F4 NAFLD groups showed sensitivity of 0.94 and p=0.0006.

### Example 3a - Clinical Application (Increasing Panel)(Increasing Values with Advanced Fibrosis)

In this example we compare values of a subject of interest with a comparator/reference subject (i.e. matched subject) with mild to moderate fibrosis (F1/F2 fibrosis).

In this example the subject with mild to moderate fibrosis has mild fibrosis.

In this example the subject of interest has F4 fibrosis.

1) A method comprising
a) providing a blood sample from a subject
b) determining the level of CPS-₁ expression in said sample
   in this example CPS-value=5.8
c) comparing the level of CPS-₁ expression of (b) to the level of CPS-₁ expression determined from a blood sample from a subject with mild to moderate fibrosis of the liver
   in this example comparing CPS-value=5.8 to CPS-value=1.2 from a subject with mild to moderate fibrosis
d) determining the level of glutamate in said sample
   in this example Glutamate value= 309
e) comparing the level of glutamate of (d) to the level of glutamate determined from a blood sample from a subject with mild to moderate fibrosis of the liver
   in this example comparing Glutamate value= 309 to Glutamate value= 78 from a subject with mild to moderate fibrosis
f) wherein if the level of CPS-₁ expression of (b) is higher than the level of CPS-₁ expression determined from a blood sample from a subject with mild to moderate fibrosis of the liver, and
   the level of glutamate of (d) is higher than the level of glutamate determined from a blood sample from a subject with mild to moderate fibrosis of the liver,
   then it is inferred that the subject has an increased likelihood of having advanced or severe (F3/F4) fibrosis of the liver.

In this example the comparisons show that the subject of interest has an increased likelihood of having advanced or severe (F3/F4) fibrosis of the liver.

In fact the subject of interest has F4 fibrosis. This shows the method of the invention in operation.

### Optional Additional Step

Patient with advanced fibrosis: Average of glutamate and CPS scores = 157

Patient with mild fibrosis: Average of glutamate and CPS scores = 39

The comparison of the averages shows a large increase in the subject of interest.

This provides an enhanced confidence that the subject of interest has an increased likelihood of having advanced or severe (F3/F4) fibrosis of the liver.

### Example 3b - Clinical Application (Decreasing Panel) (Decreasing Values with Advanced Fibrosis)

In this example we compare values of a subject of interest with a comparator/reference subject (i.e. matched subject) with mild to moderate fibrosis (F1/F2 fibrosis).

In this example the subject with mild to moderate fibrosis has mild fibrosis.

In this example the subject of interest has F4 fibrosis.

2) A method comprising
a) providing a blood sample from a subject.
b) determining the level of arginine in said sample
   in this example Arginine value =39
c) comparing the level of arginine of (b) to the level of arginine determined from a blood sample from a subject with mild to moderate fibrosis of the liver
   in this example comparing Arginine value= 39 to Arginine value= 55 from a subject with mild to moderate fibrosis
d) determining the citrulline/ornithine ratio in said sample
   in this example citrulline/ornithine ratio value = 0.3
e) comparing the citrulline/ornithine ratio of (d) to the citrulline/ornithine ratio determined from a blood sample from a subject with mild to moderate fibrosis of the liver
   in this example comparing citrulline/ornithine ratio value = 0.3 to citrulline/ornithine ratio value = 0.5 from a subject with mild to moderate fibrosis
f) determining the reserve capacity in said sample
   in this example Reserve capacity =30
g) comparing the reserve capacity of (f) to the reserve capacity determined from a blood sample from a subject with mild to moderate fibrosis of the liver
   in this example comparing Reserve capacity =30 to Reserve capacity =74 from a subject with mild to moderate fibrosis
h) wherein if the level of arginine of (b) is lower than the level of arginine determined from a blood sample from a subject with mild to moderate fibrosis of the liver, and
   the citrulline/ornithine ratio of (d) is lower than the citrulline/ornithine ratio determined from a blood sample from a subject with mild to moderate fibrosis of the liver, and
   the reserve capacity of (f) is lower than the reserve capacity determined from a blood sample from a subject with mild to moderate fibrosis of the liver,
   then it is inferred that the subject has an increased likelihood of having advanced or severe (F3/F4) fibrosis of the liver.

In this example the comparisons show that the subject of interest has an increased likelihood of having advanced or severe (F3/F4) fibrosis of the liver.

In fact the subject of interest has F4 fibrosis. This shows the method of the invention in operation.

### Optional Additional Step

Patient with advanced fibrosis: Average of arginine and citrulline/ornithine ratio and reserve capacity scores = 43

Patient with mild fibrosis: Average of arginine and citrulline/ornithine ratio and reserve capacity scores = 23

The comparison of the averages shows a large decrease in the subject of interest.

This provides an enhanced confidence that the subject of interest has an increased likelihood of having advanced or severe (F3/F4) fibrosis of the liver.

### Example 4 - Clinical Application

The values for the biomarkers taught herein were studied in a substantial cohort of subjects.

The average values across the cohorts are shown in the tables below

| Biomarker | F1-F2 cohort | F3-F4 cohort |
|---|---|---|
| Glutamate | 64 µmol/L | 137 µmol/L |
| CPS1 | 2.7 ng/ml | 4.3 ng/ml |
| Biomarker | F1-F2 cohort | F3-F4 cohort |
| Arginine | 62 µmol/L | 40 µmol/L |
| Citrulline/Ornithine Ratio | 0.41 | 0.2 |
| Reserve Capacity | 185 pmol/min | 56 pmol/min |

The average values of the F1-F2 cohort serve useful purpose as comparative values (i.e. for comparison to the values from the subject of interest, thereby advantageously avoiding having to process additional samples/additional measurements of an F1/F2 subject each time the method is carried out).

Although illustrative embodiments of the invention have been disclosed in detail herein, with reference to the accompanying drawings, it is understood that the invention is not limited to the precise embodiments shown and that various changes or modifications can be effected by one skilled in the art without departing from the scope of the invention as defined by the appended claims and their equivalents.

1) A method comprising
a) providing a blood sample from a subject
b) determining the level of CPS-₁ expression in said sample
c) comparing the level of CPS-₁ expression of (b) to the level of CPS-₁ expression determined from a blood sample from a subject with mild to moderate fibrosis of the liver
d) determining the level of glutamate in said sample
e) comparing the level of glutamate of (d) to the level of glutamate determined from ablood samplefrom a subject with mild to moderate fibrosis of the liver
f) wherein if the level of CPS-₁ expression of (b) is higher than the level of CPS-₁ expression determined from a blood sample from a subject with mild to moderate fibrosis of the liver, and the level of glutamate of (d) is higher than the level of glutamate determined from ablood sample from asubject with mild to moderate fibrosis of the liver, then it is inferred that the subject has an increased likelihood of having advanced or severe (F3/ F4) fibrosis of the liver.

2) A method comprising
a) providing a blood sample from a subject
b) determining the level of arginine in said sample
c) comparing the level of arginine of (b) to the level of arginine determined from a blood sample from a subject with mild to moderate fibrosis of the liver
d) determining the citrulline/ornithineratio in said sample
e) comparing the citrulline/ornithine ratio of (d) to the citrulline/ornithine ratio determined from a blood sample from a subject with mild to moderate fibrosis of the liver
f) determining the reserve capacity in said sample
g) comparing the reserve capacity of (f) to the reserve capacity determined from a blood samplefrom a subject with mild to moderate fibrosis of the liver
h) wherein if the level of arginine of (b) islower than thelevel of arginine determined from a blood samplefrom asubject with mild to moderate fibrosis of the liver, and the citrulline/ ornithine ratio of (d) is lower than the citrulline/ornithine ratio determined from a blood sample from asubject with mild to moderate fibrosis of the liver, and the reserve capacity of (f) is lower than the reserve capacity determined from a blood sample from a subject with mild to moderate fibrosis of the liver, then it isinferred that the subject has an increased likelihood of having advanced or severe (F3/F4) fibrosis of the liver.

3) A method according to claim 1 wherein theCPS-1 level determined is the plasma CPS-1 level.

4) A method according to claim 1 or claim 3 wherein theCPS-1 level is determined by quantitative sandwich immunoassay.

5) A method according to claim 4 wherein said quantitative sandwich immunoassay comprises an ELISA assay.

6) A method according to any of claims 1, 3, 4 or 5 wherein the level of glutamate is determined using mass spectrometry.

7) A method according to claim 2 wherein thelevel of arginine is determined using mass spectrometry.

8) A method according to claim 2 or claim 7 wherein thelevelsof citrulline/ornithine are determined using mass spectrometry.

9) A method according to claim 2, 7 or 8 wherein the reserve capacity is determined as maximal OCR minus basal respiration.

10) A method according to claim 2, 7, 8 or 9 wherein the reserve capacity is determined using the ‘XF cell mito stress test kit’ from Agilent Technologies.

11) A method according to any preceding claim wherein the subject is suspected of having liver fibrosis.

12) A method according to claim 11 wherein the subject has been previously identified as having F0-F2 liver fibrosis, preferably F1-F2 liver fibrosis.

13) A method according to any preceding claim wherein the subject is suspected of having, or has, metabolic syndrome.

14) A method according to any preceding claim wherein the subject is suspected of having, or has, diabetes, preferably type 2 diabetes.

15) A method according to any preceding claim wherein the subject is suspected of having, or has, Non-Alcoholic Fatty Liver Disease (NAFLD).

16) A method according to claim 15 wherein the subject issuspected of having, or has, Non-Alcoholic Steatohepatitis (NASH).

17) A method comprising
a) providing a first blood sample from a subject taken at afirst timepoint;
b) either
i. determining the level of CPS-₁ expression in said sample and determining the level of glutamate in said sample, or
ii. deter mining the level of arginine in said sample, determining the citrulline/ ornithine ratio in said sample and determining the reserve capacity in said sample;
c) providing a second blood sample from a subject taken at a second timepoint;
d) determining the same characteristics as were determined in step (b) for said second blood sample of step (c)
e) comparing the values from step (b) to the values from step (d);
f) inferring from the comparison of step (e) whether fibrosis has changed wherein if the values from step (b) and step (d) are different, then it is inferred that fibrosis has changed in the subject.

18) A method accordingto claim 17 wherein if thelevel of CPS-₁ expression in said second sample and the level of glutamatein said second sample are higher than the levels for said first sample, then it isinferred that fibrosis has advanced or increased in said patient, and wherein if thelevel of CPS-₁ expression in said second sample and thelevel of glutamate in said second sample are lower than the levels for said first sample, then it is inferred that fibrosis has receded or decreased in said patient.

19) A method according to claim 17 wherein if the level of argininein said second sample, the citrulline/ ornithine ratio in said second sample and the reserve capacity in said second sample are lower than the levels for said first sample, then it is inferred that fibrosis has advanced or increased in said patient, and wherein if thelevel of arginine in said second sample, the citrulline/ ornithine ratio in said second sample and the reserve capacity in said second sample are higher than the levels for said first sample, then it is inferred that fibrosis has receded or decreased in said patient.

20) A method of treating a subject with fibrosis of the liver, the method comprising performing a method according to any of claims 1 to 16 and if it is inferred that the subject has an increased likelihood of having advanced or severe (F3/F4) fibrosis of the liver, then oneor more treatments selected from the group consisting of:
reformed diet, exerciseregime, low calorie diet, and administration of GLPanalogue, such as a weekly injection of GLP analogue, is administered to said subject.
